(19) United States
(12) Patent Application Publication
(10) Pub. No.: US 2012/0220480 A1
(43) Pub. Date: Aug. 30, 2012

(54) METHOD FOR THE DETECTION OF RENAL DAMAGE

(75) Inventors: Yaremi Quiros Luis, Salamanca (ES); Laura Ferreira Redondo, Salamanca (ES); Sandra M. Sancho Martínez, Salamanca (ES); José M. Gonzalez De Buitrago Arriero, Salamanca (ES); Francisco J. Lopez Hernandez, Salamanca (ES); José M. Lopez Novoa, Salamanca (ES); Omar Garcia Sanchez, Salamanca (ES)

(73) Assignee: UNIVERSIDAD DE SALAMANCA, Salamanca (ES)

(21) Appl. No.: 13/388,506

(22) PCT Filed: Jul. 19, 2010
(86) PCT No.: PCT/EP2010/060410

§ 371 Date: May 14, 2012

(30) Foreign Application Priority Data
Aug. 4, 2009 (ES) P200930559

(51) Int. Cl. C40B 30/04; G01N 33/566

(52) U.S. Cl. 506/9; 436/501; 435/7.92

(57) ABSTRACT

The invention relates to a method for determining the presence of renal damage in an individual and also to a method for detecting one or several proteins selected from the list comprising Reg3B, fetuin B, Ras-related GTP-binding protein A serine protease inhibitor A3L, subunit 1 of COP9, gamma subunit of ATP synthase, gelsolin, ribonuclease UK114, aminoacylase 1A, alpha-enolase, keratin 5, parvalbumin alpha, ribonuclease 4 or serine protease inhibitor A3K. The renal damage may be acute renal failure. Said renal pathologies may be caused by the administration of a nephrotoxic agent, wherein the nephrotoxic agent may be an aminoglycoside antibiotic such as gentamicin, or cisplatin. The invention also provides means to differentiate the renal damage or renal failure induced by gentamicin from that induced by cisplatin, through the biochemical analysis of the urinary level of Reg3B and/or gelsolin, or fragments thereof.

The invention relates to a method for determining the presence of renal damage in an individual and also to a method for detecting one or several proteins selected from the list comprising Reg3B, fetuin B, Ras-related GTP-binding protein A, serine protease inhibitor A3L, subunit 1 of COP9, gamma subunit of ATP synthase, gelsolin, ribonuclease UK114, aminoacylase 1A, alpha-enolase, keratin 5, parvalbumin alpha, ribonuclease 4 or serine protease inhibitor A3K. The renal damage may be acute renal failure. Said renal pathologies may be caused by the administration of a nephrotoxic agent, wherein the nephrotoxic agent may be an aminoglycoside antibiotic such as gentamicin, or cisplatin. The invention also provides means to differentiate the renal damage or renal failure induced by gentamicin from that induced by cisplatin, through the biochemical analysis of the urinary level of Reg3B and/or gelsolin, or fragments thereof.

### BACKGROUND ART

Aminoglycoside antibiotics are extensively used against bacterial infections. Specifically, gentamicin is used against Gram negative infections. Its therapeutic use and efficacy is seriously hindered by its toxicity, which mainly occurs on a renal and auditory level (Martinez-Salgado C, Lopez-Hernandez FJ and Lopez-Novoa J M. 2007, Toxicol Appl Pharmacol., 223: 86-98). Gentamicin-induced nephrotoxicity appears in 10-25% of treatments (Leehey D J et al., 1993, J. Am. Soc. Nephrol., 4: 81-90). It is mainly characterised by tubular damage (Nakakuki M et al., 1996, Can J Physiol Pharmacol., 74: 104-111), but glomerular (Martinez-Salgado C, Lopez-Hernandez FJ and Lopez-Novoa J M. 2007, Toxicol Appl Pharmacol., 223: 86-98) and vascular (Goto T et al., 2004, Virchows Arch., 444: 362-74; Seqilmi§ M A, et al., 2005, Nephron Physiol., 100: 13-20) alterations can also appear in a dose-dependent manner (Hishida A et al., 1994, Ren Fail., 16: 109-116). The tubular damage mainly affects the proximal tubule, wherein, depending on the intensity of the aggression, the lesion can range from minor epithelial scaling to more severe tubular necrosis (Nakakuki et al., 1996. Can J Physiol Pharmacol., 74: 104-111). Tubular lesions produce an imbalance in the reabsorption capacity that activates tubuloglomerular feedback, which drastically reduces the glomerular filtration rate (GFR) in order to prevent a massive loss of fluid. Finally, gentamicin reduces renal blood flow (RBF) by contraction of the preglomerular arteries as well as the afferent and efferent arterioles (Klotman et al., 1983. Kidney Int., 24: 638-643). As a result of a lower RBF, GFR deteriorates. A lower RBF also contributes to tissular necrosis, especially inside the cortical zone (Cheung et al., 2008. Drugs Aging, 25: 455-476).

Depending on the age, sex, previous renal function, duration of treatment, therapeutic regimen, hydration level and other concomitant states (for example, pregnancy or hypothyroidism), gentamicin-related nephrotoxicity can sometimes lead to acute renal failure.

Acute renal failure is a type of renal damage characterized by loss of the kidney's excretory function, sufficient to prevent clearing the blood of waste products and water as well as maintaining the electrolytic balance (Bellomo R, Kellum JA and Ronco C, 2007. Intensive Care Med., 33: 409-413). Acute kidney injury and acute renal failure can be induced by a wide range of aggressions including drugs, chemical poisons, hypoxia, urinary tract obstruction, infections and others (Binswanger U, 1997. Kidney Blood Press Res., 20: 163). This type of renal damage represents an enormous human and socioeconomic burden as a result of its high incidence and mortality rate. It is estimated that almost 1% of hospital admissions are related to acute renal failure, and approximately 2-7% of hospitalised patients eventually develop it. More importantly, the mortality rate among patients with acute renal failure remains remarkably high, at approximately 50% of cases.

In clinical practice, acute renal failure (and, in general, acute kidney injury) is diagnosed when the renal dysfunction produces measurable symptoms. These are typically based on determining creatinine and urea levels. Most commonly their concentration in serum increases as the GFR decreases. However, in the state where high urea and creatinine serum levels are already observed, acute renal failure is difficult to treat. Thus, the current trends in diagnosis seek to detect incipient physiopathological events occurring in early stages, when the damage is less extensive (Vaidya V S, Ferguson M A and Bonventre J V, 2008. Rev Pharmacol Toxicol., 48:463-493). Among these, measuring certain cellular enzymes present in urine as a result of the breakdown of tubular cells is currently the most refined procedure for the early detection of acute kidney injury, which courses with tubular damage. These enzymes include N-acetyl-D-glucosaminidase (NAG), but also others such as lactate dehydrogenase (LDH), alkaline phosphatase (ALP) or gamma glutamyl transpeptidase (GGT). Most of these enzymes have a moderate value as early and sensitive urinary markers of acute kidney injury, mainly due to problems of stability and inhibition by other urine components (Vaidya V S, Ferguson M A and Bonventre J V, 2008. Rev Pharmacol Toxicol., 48: 463-493). Latest generation early markers include, among others, urine measurements of kidney injury molecule 1 (KIM-1), or neutrophil gelatinase-associated lipocalin (NGAL) (Vaidya et al., 2008).

However, there are still aspects that can be improved in the diagnosis of acute kidney injury and acute renal failure. One of these aspects is the aetiological diagnosis of the damage, in other words, not only detecting the damage early, but also detecting its cause. This aspect becomes particularly important in clinical situations where different potentially nephrotoxic agents converge in the same individual at the same time, like for example various potentially harmful drugs for the kidneys. Under these circumstances, when the first symptoms of nephrotoxicity appear, it is impossible to know using current diagnosis technology which of the drugs causes the damage, preventing the replacement or change in therapeutic regime of only that drug without altering that of the other drugs. Among the drugs of this type, one would highlight aminoglycoside antibiotics. Being able to identify in this way specific markers or collections of markers for each potentially nephrotoxic agent would make it possible to provide a more rational, individualised and specific treatment in daily clinical situations such as simultaneous treatments containing two or more potentially nephrotoxic drugs. This would allow the treatment to be redirected correctly by replacing or changing the therapeutic regime of the harmful drug.

### SUMMARY OF THE INVENTION

The invention relates to a method for determining renal damage in an individual as well as a method for predicting the progression of said renal damage by means of detecting one or several proteins selected from the list comprising Reg3B, fetuin B, Ras-related GTP-binding protein A, serine protease inhibitor A3L, subunit 1 of COP9, gamma subunit of ATP synthase, gelsolin, ribonuclease UK114, aminoacylase 1A, alpha-enolase, keratin 5, parvalbumin alpha, ribonuclease 4 or serine protease inhibitor A3K. The renal damage may be acute renal failure. Said renal pathologies may be caused by the administration of a nephrotoxic agent, wherein the nephrotoxic agent may be an aminoglycoside antibiotic such as gentamicin, or cisplatin.

The present invention provides evidence that renal damage or acute renal failure induced for example, but without limitation, by gentamicin, is related to an increase in the excretion of any protein selected from the list mentioned in the previous paragraph, or any combination thereof.

Therefore, the present invention provides tools for detecting renal damage or acute renal failure. These tools allow the progression of renal damage or acute renal failure to be predicted, in other words, to supervise the progression of said pathology when the person is treated with, for example, but without limitation, therapeutic substances, or when the person is exposed to any nephrotoxic or non-nephrotoxic agent or condition.

Also, the present invention provides a notable advantage: the detection of a protein, or any fragment thereof, which is selected from the list comprising Reg3B, fetuin B, Ras-related GTP-binding protein A, serine protease inhibitor A3L (serpin A3L), subunit 1 of COP9, gamma subunit of ATP synthase, gelsolin, ribonuclease UK114, aminoacylase 1A, alpha-enolase, keratin 5, parvalbumin alpha, ribonuclease 4 or serine protease inhibitor A3K (serpin A3K), in urine samples, entails an additional advantage for the patient since its evacuation bodily fluid is a normally-occurring physiological necessity. This means that sampling from the individual is non-aggressive.

Finally, the present invention provides means to detect and distinguish whether the renal damage or renal failure is caused by gentamicin or cisplatin. This is another notable diagnostic advantage to discern the cause of renal damage or renal failure in polymedicated patients, in order to properly reshape their treatments.

A brief description follows of the proteins detected and/or quantified in the method of the present invention.

Regenerating islet-derived protein 3 beta (REG3β, REG-III o Reg III β), is also known as, among other synonyms as pancreatic stone protein 2, pancreatitis-associated protein (Pap), Pancreatitis-associated protein 1 (Pap1), HIP, or INGAP. This protein has been related to lectin. Reg3B is present in small amounts in a normal pancreas but is overexpressed in the acute phase of pancreatitis. It can be involved in the response for controlling bacterial proliferation during acute pancreatitis. It has been possible to clone it.

Fetuin B (also known as, among others, 16G2, Fetuin-B precursor, Gugu, IRL685). Fetuin is a protein synthesised in the kidney and secreted into the blood stream. It belongs to a large group of proteins which facilitate the transport and availability of a wide variety of substances in the blood stream. This protein is more abundant in foetal blood than in that of an adult individual, hence the name “fetuin”.

Ras-related GTP-binding protein A (also known as, among others, Rag A, FIP1, FIP-1, RagA, RAGA, RRAGA, Rag A, or Adenovirus E3 14.7 kDa-interacting protein 1). This protein may be in the form of a homodimer. It participates in the RCC1/Ran-GTPase signalling pathway and can perform a direct function in TNF-alpha-mediated signalling, which brings about the induction of cellular death. It is expressed ubiquitously in skeletal muscle, heart and brain.

The protein serpin A3L (some synonyms to refer to this protein include serine protease inhibitor A3L, serine protease inhibitor 1 or contratripsin-like protease inhibitor 3), belongs to a group of proteins capable of inhibiting other enzymes of the protease group. The name Serpin comes from the combination of Serine protease inhibitor by virtue of its functional properties. Serpin A3L is induced by growth hormones and a reduction in its expression levels in rats has been described during acute inflammation. The product of the gene's expression has been located in the liver of Rattus novergicus (rat). The protein serpin A3K (some other synonyms to refer to this protein include contratripsin-like protease inhibitor 1, Kallikrein-binding protein, serine protease inhibitor 2 or growth hormone-regulated proteinase inhibitor) is an extracellular serpin protein that has demonstrated low levels in the retina of rats suffering from diabetes, which can contribute to a retinopathy.

The signalosome COP9 is a protein complex conserved in eukaryote cells composed of eight subunits (CSN1 to CSN8). COP9 is an ubiquitination regulator. Ubiquitination consists of a protein marking process using the protein ubiquitin for proteolysis. Ubiquitin anchors to the protein to be eliminated and in this way, the marked protein moves towards the proteasome, a structure where the proteolysis process is carried out.

The gamma subunit of the ATP-synthase complex (also known as the gamma subunit of F-type ATPase) forms the central axis that connects the F0 rotary domain to the F1 catalytic domain of the complex. The protein ATP synthase produces ATP using ADP in the presence of a proton gradient between the cell outside and inside. Type-F ATPases have two components, the F1 component, with a catalytic function, and the F0 component, which is the proton channel embedded in the membrane. F1 has five subunits: alpha, beta, gamma, delta and epsilon. F0 has three main subunits: a, b and c. The gamma subunit is important in regulating the activity of the complex and the flow of protons.

Gelsolin is a globular protein of 82 KDa with six sub-domains, named S1-S6. This protein is involved in the assembly of actin filaments.

Ribonuclease (RNase) is an enzyme (nuclease) which catalyses the hydrolysis of RNA into smaller components. Ribonuclease UK114 is responsible for translation inhibition, hydrolysing the mRNA. Ribonuclease 4 (RNase 4) is a protein of approximately 16 kDa with a preference for the hydrolysis of ribonucleic acid polymers.

The enzyme aminoacylase 1 (Acy 1) is located in cytosol, is homodimeric, dependent on its zinc bond and catalyses the hydrolysis of acylated L-amino acids.

The enzyme alpha-enolase, a well-known glycolytic enzyme, has been identified as an autoantigen in Hashimoto's encephalopathy, and has also been related to severe asthma. The reduced expression of the enzyme has been found in the cornea of people suffering from keratoconus.

Keratin 5 is a cytokeratin frequently related to keratin 14. Type-II cytokeratins consist of basic or neutral proteins that are organised into pairs of chains different from keratin co-expressed during the differentiation of simple and stratified epithelial tissues. Mutations in these genes have been associated with diseases known as simple epidermolysis.

The protein parvalbumin is a low molecular weight albumin (normally 9-11 kDa) which needs to bind to calcium in order to carry out its function. It is structurally related to calmodulin and troponin-C. Parvalbumin is located in fast-contracting muscles as well as in the brain and in some endocrine tissues.

A first aspect of the present invention relates to a method to provide useful data for determining renal damage, comprising:

a. obtaining an isolated biological sample from an individual, and
b. detecting and/or quantifying in the sample obtained in (a) at least one protein, or any combination thereof, selected from the list comprising:

a protein having at least 60% identity in respect of the amino acid sequence SEQ ID NO: 1 or SEQ ID NO: 2, or any fragments thereof,

a protein having at least 80% identity in respect of the amino acid sequence SEQ ID NO: 3 to 11, or any fragments thereof, or

a protein having at least 90% identity in respect of the amino acid sequence SEQ ID NO: 12 to 14, or any fragments thereof.

The term “renal damage” as understood in the present invention relates to any damage in the renal system of an individual that may or may not cause a condition in the individual that allows said condition to be attributed to a renal illness. The origin of said renal damage may be, for example, but without limitation, genetic, immune, ischemic, or treatment with any drug. The renal damage or disease referred to above may also be produced by a surgical procedure, for example, but without limitation, of the kidney, prostate, bladder, ureter or urethra.

Below is a description of the correspondence between the sequences mentioned and the proteins described, including each one's access number (in brackets) and the organism it comes from:

SEQ ID NO: 1; Reg3B (A49616, corresponding to Homo sapiens),

SEQ ID NO: 2; fetuin B (NP_{—}055190.2, corresponding to Homo sapiens),

SEQ ID NO: 3; serine protease inhibitor A3K (P05545.3, corresponding to Rattus novergicus),

SEQ ID NO: 4; gamma subunit of ATP synthase (NP_{—}001001973.1, corresponding to Homo sapiens),

SEQ ID NO: 5; gelsolin (NP_{—}000168.1, corresponding to Homo sapiens),

SEQ ID NO: 6; ribonuclease UK114 (P52758.1, corresponding to Homo sapiens),

SEQ ID NO: 7; aminoacylase 1A (NP_{—}000657.1, corresponding to Homo sapiens),

SEQ ID NO: 8; alpha-enolase (AAB88178.1, corresponding to Homo sapiens),

SEQ ID NO: 9; keratin 5 (NP_{—}000415.2, corresponding to Homo sapiens),

SEQ ID NO: 10; parvalbumin alpha (P20472.2, corresponding to Homo sapiens),

SEQ ID NO: 11; ribonuclease 4 (AAA96750.1, corresponding to Homo sapiens),

SEQ ID NO: 12; serine protease inhibitor A3L (P05544.3, corresponding to Rattus norvegicus)

SEQ ID NO: 13; subunit 1 of COP9 (Q13098.4, corresponding to Homo sapiens),

SEQ ID NO: 14; Ras-related GTP-binding protein A (NP_{—}006561.1, corresponding to Homo sapiens).

The percentage of identity has been established based on determining the % of identity of the amino acid sequence of the protein corresponding to Homo sapiens in respect of the amino acid sequence of the protein corresponding to Rattus norvegicus (Table 1).

TABLE 1
Percentages of identity of the proteins of the invention corresponding
to Rattus norvegicus and Homo sapiens.

| | | Access No. | | |
|---|---|---|---|---|
| | | Rattus | Access No. | % of |
| Protein | SEQ ID NO | norvegicus | Homo sapiens | Identity |
| Reg3B | 1 | P25031 | A49616 | 69 |
| Fetuin B | 2 | Q9QX79 | NP_055190.2 | 62 |
| Serine protease | 3 | P05545.3 | CAD83829.1 | 88.2 |
| inhibitor A3K | | | | |
| (Serpin A3K) | | | | |
| Gamma subunit | 4 | P35435 | NP_001001973.1 | 91.2 |
| of ATP | | | | |
| synthase | | | | |
| Gelsolin | 5 | Q68FP1 | NP_000168.1 | 93.1 |
| Ribonuclease | 6 | P52759.3 | P52758.1 | 87.6 |
| UK114 (RNase | | | | |
| UK114) | | | | |
| Aminoacylase | 7 | Q6AYS7.1 | NP_000657.1 | 87.7 |
| 1A (Acy 1A) | | | | |
| Alpha-enolase | 8 | P04764 | AAB88178.1 | 94.6 |
| Keratin 5 | 9 | Q6P6Q2 | NP_000415.2 | 88.3 |
| Parvalbumin | 10 | P02625.2 | P20472.2 | 91.8 |
| alpha | | | | |
| Ribonuclease 4 | 11 | O55004 | AAA96750.1 | 80.5 |
| (RNase 4) | | | | |
| Serine protease | 12 | P05544.3 | CAD83829.1 | 99.3 |
| inhibitor A3L | | | | |
| (Serpin A3L) | | | | |
| Subunit 1 | 13 | P97834.1 | Q13098.4 | 96.8 |
| of COP9 | | | | |
| Ras-related | 14 | Q63486 | NP_006561.1 | 100 |
| GTP-binding | | | | |
| protein A | | | | |

The term “% of identity” between two amino acid sequences, as understood in the present invention, refers to the number of amino acid positions over the total length of the sequence under comparison, wherein all the amino acids in that position are identical.

More preferably, in step (b) of the method at least one protein, or any combination thereof, is detected and/or quantified, having:

at least 60, 65, 70, 75, 80, 85, 90 or 95% identity in respect of the amino acid sequence SEQ ID NO: 1 or SEQ ID NO: 2, or any fragments thereof,

at least 80, 85, 90 or 95% identity in respect of amino acid sequence SEQ ID NO: 3 to 11, or any fragments thereof, or

at least 90 or 95% identity in respect of the amino acid sequence SEQ ID NO: 12 a 14, or any fragments thereof.

Hereinafter, when reference is made to any of the proteins of the present invention it shall be taken into account that any protein can also be detected having the percentage of identity mentioned in the preceding paragraphs, according to the protein in question. Therefore, hereinafter, any of the proteins of the present invention may be referred to as “protein of the invention” or “protein of the present invention”.

In order to detect and/or quantify the protein of the present invention it is sufficient to detect one or more fragments of said protein since said fragment is a constituent of the amino acid sequence and of the structure of the protein.

Step (b) of the method of the present invention relates to the detection and quantification of the protein, or any fragment thereof, or also relates to its detection or to its quantification. Said detection and/or quantification may be carried out by means of any technique known in the state of the art.

At the same time, as already mentioned, the method of the present invention can be carried out through the detection and/or quantification of the combination of any of the proteins of the abovementioned list or the combination of any of the fragments of said proteins. Moreover, the term “any combination thereof” also refers to the fact that one or more proteins of the invention (or any of their fragments) may be detected in combination with the detection of any other protein different to any of the proteins of the present invention.

Any of the proteins of the present invention is the product of the expression of a nucleotide sequence. This nucleotide sequence may be, for example, but without limitation, any RNA, for example, but without limitation, messenger RNA (mRNA), or any fragment thereof. The nucleotide sequence may also be complementary DNA (cDNA) or any fragment thereof. cDNA is a complementary DNA of a mRNA or is also the nucleotide sequence that comprises the exons but not the introns of the genomic nucleotide sequence, in other words, cDNA is the encoding sequence. Transcription of the genomic nucleotide sequence of the gene encoding the protein and of its cDNA encodes the same mRNA and, therefore, the same protein. In the present invention it is also possible to detect any RNA or any DNA, or any fragment thereof, instead of detecting the protein, or simultaneously.

A preferred embodiment relates to a method to provide useful data for determining renal damage, wherein at least one protein is detected and/or quantified, or any combination thereof, selected from the list comprising: SEQ ID NO: 1 to 14, or any fragment thereof.

Another preferred embodiment relates to a method to provide useful data for determining renal damage, wherein a protein is detected and/or quantified having at least 60% identity in respect of SEQ ID NO: 1 and/or a protein having at least 60% identity in respect of SEQ ID NO: 2, or any fragments thereof. According to a more preferred embodiment, the protein SEQ ID NO: 1 and/or SEQ ID NO: 2 is detected and/or quantified, or any fragments thereof.

Another preferred embodiment of the present invention refers to method to provide useful data for determining renal damage, wherein is detected and/or quantified a protein at least 60% identical to SEQ ID NO: 1, or any fragment thereof. According to a more preferred embodiment, the protein SEQ ID NO: 1 is detected and/or quantified, or any fragments thereof.

Another preferred embodiment of the present invention refers to method to provide useful data for determining renal damage, wherein is detected and/or quantified a protein at least 80% identical to SEQ ID NO: 5, or any fragment thereof. According to a more preferred embodiment, the protein SEQ ID NO: 5 is detected and/or quantified, or any fragment thereof.

Another preferred embodiment of the present invention refers to a method to provide useful data for determining renal damage, wherein is detected and/or quantified the protein SEQ ID NO: 1 and the protein SEQ ID NO: 5, or any fragment thereof.

Another preferred embodiment relates to a method to provide useful data for determining renal damage, wherein it additionally comprises comparing the data obtained in step (b) with data obtained from control samples in order to discover any significant deviation.

The term “control samples” as understood in the present invention refers, for example, but without limitation, to a sample obtained from an individual who does not suffer from renal damage (a healthy individual). This type of control sample is a negative control sample or negative control for renal damage.

The term “significant deviation” as understood in the present invention relates to the presence of the protein in the isolated sample, or a higher concentration of the protein of the invention in the isolated sample in respect of an isolated sample from a healthy individual. The healthy individual is selected by means of measuring the level of one or more common markers of renal damage. The common marker is selected from a list comprising, but without limitation, creatinine, blood urea nitrogen (BUN) or proteinuria.

The biological sample isolated from an organism, such as for example, but without limitation, a human or other animal, may be a bodily fluid or any cellular tissue from said organisms.

Another preferred embodiment of the present invention relates to a method for determining renal damage which comprises the steps of the methods of obtaining data described in previous paragraphs and additionally comprises the attribution of the significant deviation to the development of said renal damage in the individual. Consequently, this preferred embodiment is a method for diagnosing renal damage.

Another preferred embodiment relates to the method to provide useful data for determining renal damage wherein said renal damage is acute renal failure. The term “acute renal failure”, as understood in the present invention, refers to an acute renal failure in any stage (or severity) of renal dysfunction. The origin of the acute renal failure may be, for example, but without limitation, genetic, immune, ischemic, or a treatment with any drug. The acute renal failure may also be produced by a surgical procedure, for example, but without limitation, of the kidney, prostate, bladder, ureter or urethra. The terms “acute kidney injury” and “renal damage”, as understood in the present invention, refers to and acute damage to the kidneys, of any intensity, which may lead or not to an acute renal failure.

In another preferred embodiment of the method to provide useful data for determining renal damage or acute renal failure, the biological sample of step (a) is a bodily fluid. The bodily fluid may include fluids excreted or secreted by the animal body as well as fluids that are not excreted or secreted. The bodily fluid is selecting from a list comprising, but without limitation, amniotic fluid surrounding the foetus, aqueous humour, blood, plasma, interstitial fluid, lymph, milk, mucus (including nasal secretion and phlegm), saliva, sebum (skin oil), serum, sweat, tears or urine. In a more preferred embodiment, the bodily fluid is serum. The protein of the present invention may be found in any biological compartment present in the aforesaid bodily fluid such as for example, but without limitation, a cell or a vesicle. In a more preferred embodiment, the bodily fluid is urine.

Another preferred embodiment relates to the method to provide useful data for determining renal damage or acute renal failure, wherein the protein, or any fragment thereof, is detected and/or quantified by means of electrophoresis, immunoassay, chromatography and/or microarray technology.

The detection and/or quantification of the protein of the present invention may be carried out by means of any of the aforesaid techniques or by any combination thereof. The protein may be detected by evaluating its presence or absence. Detection may be carried out by specific recognition of any fragment of protein by means of any probe and/or any antibody. The protein of the present invention, or any fragment thereof, may be quantified, with these data serving as reference for comparison with the data obtained from the control sample and for finding any significant deviation. This significant deviation may be attributed to the diagnosis of renal damage in the individual from which the problem sample originates. In a preferred embodiment, the protein of the invention, or any fragment thereof, may be detected and/or quantified by means of electrophoresis and/or immunoassay.

Electrophoresis is an analytical technique of separation based on the movement or migration of macro-molecules dissolved in a medium (electrophoresis buffer) through a matrix or solid support as a result of the action of an electrical field. The behaviour of the molecule will depend on its electrophoretic mobility and this mobility depends on the load, size and shape. There are numerous variations of this technique based on the equipment used, supports and conditions for carrying out the protein separation. Electrophoresis is selected from the list comprising, but without limitation, capillary electrophoresis, electrophoresis on paper, electrophoresis in agarose gel, electrophoresis in polyacrylamide gel, isoelectrofocus or two-dimensional electrophoresis.

An immunoassay is a biochemical test that measures the concentration of a substance in a biological liquid using the reaction of an antibody or antibodies with any of its antigens. The assay makes use of the specificity of an antibody for its antigen. The amount of antibody or antigen can be detected by means of methods known in the state of the technique. One of the most common methods is the one based on marking the antigen or the antibodies. This marking can be carried out, but without limitation, by an enzyme, radioisotopes (radioimmunoassay), magnetic labels (magnetic immunoassay) or fluorescence, and also by other techniques including agglutination, nephelometry, turbidimetry or Western Blot. Heterogeneous immunoassays may be competitive or non-competitive. The immunoassay may be competitive: the response will be inversely proportional to the concentration of the antigen in the sample, or may be non-competitive (also known as “sandwich assay”): the results are directly proportional to the concentration of the antigen. An immunoassay technique that can be used in the present invention is the ELISA assay (Enzyme-Linked ImmunoSorbent Assay).

Using chromatography techniques, molecules can be separated, but without limitation, according to load, size, molecular mass, polarity or redox potential. The chromatography technique is selected, but without limitation, from liquid chromatography (partition chromatography, adsorption chromatography, exclusion chromatography or ion-exchange chromatography), gas chromatography or supercritical fluids chromatography.

The microarray technology of the present invention is based, for example, on fixing a molecule that recognises the protein of the present invention on a solid support. The antibody-based microarray is the most common type of protein microarray. In this case, the antibodies are fixed on the solid support (the term chip can also be used to refer to a microarray). These antibodies are used to capture molecules that allow detection of the relevant proteins, but without limitation, of biological samples, cell lysates, serum or urine. The term “solid support” as used in the present invention refers to a wide variety of materials, for example, but without limitation, ion exchange or adsorption resin, glass, plastic, latex, nylon, gel, cellulose esters, paramagnetic spheres or the combination of any of these.

According to another preferred embodiment of the method to provide useful data for determining renal damage or acute renal failure of the present invention, the renal damage or acute renal failure is produced by the administration of or by the individual's exposure to at least one nephrotoxic agent. A more preferred embodiment relates to the method wherein the nephrotoxic agent is an aminoglycoside antibiotic.

This nephrotoxic agent may cause one or more renal pathologies due to the level of administration or exposure. Said administration or exposure may occur over a period of time or may be limited to a single event, and may also be due to one or several compounds. The circumstances of exposure may be involuntary, accidental, intentional, a result of an overdose or the result of a therapeutic need (administration). The kidney is the main excretory organ because it maintains the homeostasis of water-soluble molecules and can actively concentrate certain substances. In general, the proximal, distal or urinary tubule may be repaired, but the glomerules and medulla are significantly less prone to repair.

The nephrotoxic agent, when administered, may be a pharmaceutical composition (therapeutic substance) or, but without limitation, a halogenated anaesthetic or a compound included in a functional food, or in a vitamin supplement or in a nutritional supplement. The nephrotoxic agent, when the individual is exposed, may be also, a chemical such as, but without limitation, a heavy metal, plaguicide or antimicrobial agent. The plaguicide may be, but without limitation, a fungicide, herbicide, insecticide, algaecide, molluscicide, acaricide or raticide. The antimicrobial agent may be, but without limitation, a bactericide, an antibiotic, an antibacterial agent, antiviral agent, antifungal agent, antiprotozoal agent or antiparasitic agent.

The aminoglycoside antibiotic (aminoglycoside) acts by binding to the bacterial ribosomal subunits 30S and 50S, inhibiting the translocation of peptidyl-tRNA and also causing an erroneous reading of mRNA, leaving the bacteria incapable of synthesising vital proteins for its growth. The aminoglycoside antibiotic may be for example, but without limitation, amikacin, arbekacin, gentamicin, kanamycin, neomycin, netilmicin, paromomycin, rhodostreptomycin, streptomycin, tobramycin, apramycin, spectinomycin, hygromycin B, verdamicin, astromicin or puromycin. According to an even more preferred embodiment, the aminoglycoside antibiotic is gentamicin.

Gentamicin is a broad-spectrum aminoglycoside antibiotic commonly used for treating Gram-negative aerobic bacterial infections. Aminoglycosides are poorly absorbed when orally administered, but are rapidly eliminated by the kidneys. On a separate note, aminoglycosides spread in bacterial cells through channels located in the outer membrane and are transported to the cytoplasm. As a result, gentamicin acts by interfering with the synthesis of bacterial proteins but can cause renal damage in the proximal tubule, particularly in segment S1 or S2, which can induce an acute kidney injury, which can give rise to the development of an acute renal failure. The acute renal failure may be due, additionally, to the simultaneous or sequential administration of a second nephrotoxic agent. This second compound may be for example, but without limitation, uranyl nitrate or cisplatin. Uranyl nitrate is a nephrotoxic agent that causes severe renal insufficiency and acute tubular necrosis. Other target organs include the liver, lungs or brain. Cisplatin is a broad-spectrum antitumoral agent commonly used to treat tumours of the testicles, ovaries, bladder, skin, neck or lungs. Cisplatin spreads in cells and functions by interspersing inside and between DNA chains, causing the cell's death.

Another embodiment of the present invention refers to the method to provide useful data for determining renal damage or acute renal failure, wherein the protein is detected from 12 hours after the beginning of the administration of or exposure to the nephrotoxic agent. According to a more preferred embodiment, the protein is detected from 24 hours after the beginning of the administration of or exposure to the nephrotoxic agent.

Another embodiment of the present invention refers to the method to provide useful data for determining renal damage or acute renal failure, for determining the cause of the renal damage or of the renal failure, differentiating said renal damage or said renal failure caused by gentamicin from that caused by cisplatin by means of detecting and/or quantifying SEQ ID NO: 1 and/or SEQ ID NO: 5, or any fragments thereof. Another more preferred embodiment of the present invention refers to the method to differentiate or discern said renal damage or said renal failure caused by gentamicin from that caused by cisplatin, wherein

a. If it is detected or quantified SEQ ID NO: 1 and/or SEQ ID NO: 5 the cause of the renal damage or renal failure is the administration or exposure to gentamicin.

b. If it is not detected and/or quantified SEQ ID NO: 1 and/or SEQ ID NO: 5, the cause of the renal damage or renal failure is the administration or exposure to cisplatin.

Therefore, the method is useful to determine differentially the cause of the renal damage or of the renal failure. The method can discriminate if the renal damage or the renal failure is due to the administration or exposure to gentamicin or to cisplatin. In this case, there are several possibilities:
(i) If it is detected and/or quantified SEQ ID NO: 1 and SEQ ID NO: 5, or if it is detected and/or quantified SEQ ID NO: 1, or If it is detected and/or quantified SEQ ID NO: 5, the cause of the renal damage or renal failure is the administration or exposure to gentamicin.
(ii) If it is not detected and/or quantified SEQ ID NO: 1 and SEQ ID NO: 5, or if it is not detected and/or quantified SEQ ID NO: 1, or if it is not detected and/or quantified SEQ ID NO: 5, the cause of the renal damage or renal failure is the administration or exposure to cisplatin.

Another aspect of the present invention is a method for predicting the progress of renal damage due to the administration of at least one nephrotoxic agent, which comprises, in the first place (a) determining a first concentration of a protein, or any fragment thereof, selected from the list comprising:

a protein having at least 60% identity in respect of amino acid sequence SEQ ID NO: 1 or SEQ ID NO: 2, or any fragments thereof,

a protein having at least 80% in respect of amino acid sequence SEQ ID NO: 3 to 11, or any fragments thereof, or

a protein having at least 90% identity in respect of the amino acid sequence SEQ ID NO: 12 to 14, or any fragments thereof,

in a bodily fluid isolated from an individual exposed or not to a nephrotoxic agent. In the second place, a second concentration is determined of the protein whose concentration has been determined in (a), in a bodily fluid of the individual, after determining the first concentration of said protein in the exposed individual, or after starting the administration or exposure to the nephrotoxic agent in the non-exposed individual, and (c) comparing the second concentration obtained in step (b) with the first concentration contained in step (a) in order to find any significant deviation. In other words, if the first concentration is determined from the sample of an individual exposed to the nephrotoxic agent, the second concentration will be measured after determining the first concentration, and if the first concentration is determined from the sample of an individual not exposed to the nephrotoxic agent, the second concentration will be measured after starting the individual's administration or exposure to the nephrotoxic agent. The second concentration is compared to the first concentration looking for any significant deviation. The significant deviation may be in the sense of higher or lower values when the second concentration is compared to the first concentration, or when any significant deviation is compared to a previous determination of the concentration.

In the present invention, the term “predicting the progress” refers to a conclusion from monitoring or supervising the progress of the renal damage, in other words, the declaration of the progress of renal damage due to the administration of at least one nephrotoxic agent.

A preferred embodiment relates to the method for predicting the progress of renal damage due to the administration of at least one nephrotoxic agent, wherein in steps (a) and (b) at least one protein is detected and/or quantified, or any combination thereof, selected from the list comprising: SEQ ID NO: 1 to 14, or any fragment thereof.

Another preferred embodiment relates to the method for predicting the progress of renal damage due to the administration of at least one nephrotoxic agent, wherein the concentration is determined of a protein having at least 60% identity in respect of SEQ ID NO: 1 and/or a protein having at least 60% identity in respect of SEQ ID NO: 2, or any fragments thereof. According to a more preferred embodiment, the protein SEQ ID NO: 1 and/or SEQ ID NO: 2 is detected and/or quantified, or any fragment thereof.

Another preferred embodiment of the present invention refers to method for predicting the progress of renal damage due to the administration of at least one nephrotoxic agent, wherein is detected and/or quantified a protein at least 60% identical to SEQ ID NO: 1, or any fragment thereof. According to a more preferred embodiment, the protein SEQ ID NO: 1 is detected and/or quantified, or any fragments thereof.

Another preferred embodiment of the present invention refers to method for predicting the progress of renal damage due to the administration of at least one nephrotoxic agent, wherein is detected and/or quantified a protein at least 80% identical to SEQ ID NO: 5, or any fragment thereof. According to a more preferred embodiment, the protein SEQ ID NO: 5 is detected and/or quantified, or any fragment thereof.

Another preferred embodiment of the present invention refers to method for predicting the progress of renal damage due to the administration of at least one nephrotoxic agent, wherein is detected and/or quantified the protein SEQ ID NO: 1 and the protein SEQ ID NO: 5, or any fragment thereof.

Another preferred embodiment relates to the method for predicting the progress of renal damage due to the administration of at least one nephrotoxic agent, wherein the renal damage is an acute renal failure.

According to another preferred embodiment of the method for predicting the progress of renal damage or an acute renal failure, the nephrotoxic agent is an aminoglycoside antibiotic. A more preferred embodiment relates to the method wherein the aminoglycoside antibiotic is gentamicin.

According to another preferred embodiment of the method for predicting the progress of renal damage or an acute renal failure, the nephrotoxic agent is cisplatin.

Another preferred embodiment relates to the method for predicting the progress of renal damage or an acute renal failure, wherein the bodily fluid is urine or serum.

In order to refer to any of the methods of the present invention for obtaining useful data for determining renal damage or an acute renal failure, or any of the methods for predicting the progress of the aforesaid renal damage or aforesaid acute renal failure, the term “method/s of the present invention” or “method/s of the invention” may be used.

According to a preferred embodiment of the method of the present invention, the individual is a human being. The individual of the method of the invention may be an animal, since the method in question is useful for veterinary purposes.

Another aspect of the present invention is the use of at least one protein, or any combination thereof, selected from the list comprising:

a protein having at least 60% identity in respect of amino acid sequence SEQ ID NO: 1 or SEQ ID NO: 2, or any fragments thereof,

a protein having at least 80% identity in respect of amino acid sequence SEQ ID NO: 3 to 11, or any fragments thereof, or

a protein having at least 90% identity in respect of the amino acid sequence SEQ ID NO: 12 to 14, or any fragments thereof,

as a biomarker for determining renal damage or for predicting the progress of renal damage.

A preferred embodiment relates to the use wherein at least one protein is detected and/or quantified, or any combination thereof, selected from the list comprising SEQ ID NO: 1 to 14, or any fragment thereof.

Another preferred embodiment relates to the use wherein the concentration of a protein having at least 60% identity in respect of SEQ ID NO: 1 is determined and/or a protein having at least 60% identity in respect of SEQ ID NO: 2, or any fragments thereof. According to a more preferred embodiment, the protein SEQ ID NO: 1 and/or SEQ ID NO: 2 are determined and/or quantified, or any fragment thereof.

Another preferred embodiment relates to the use, wherein the protein is selected from the list comprising SEQ ID NO: 1 to 14, or a fragment thereof.

According to another preferred embodiment of the use of the present invention, is selected a protein with at least 60% identical to SEQ ID NO: 1, or a fragment thereof. Preferably is selected the protein SEQ ID NO: 1, or a fragment thereof.

Another preferred embodiment of the present invention refers to the use, wherein is selected a protein with at least 80% identical to SEQ ID NO: 5, or a fragment thereof, as biomarker for determining the risk of developing the renal damage, or for determining the renal damage, or for predict the progression of a renal damage. Preferably is selected the SEQ ID NO: 5, or a fragment thereof.

Another preferred embodiment of the present invention refers to the use, wherein the renal damage is acute renal failure.

This biomarker indicates a change in the expression or state of a protein related to renal damage or to the progress of renal damage. Once the bioindicator has been validated, it can be used to diagnose renal damage, the progress of renal damage or to adjust an individual's treatments for such renal damage, for example, treatment using various drugs or regimes of administration.

If a treatment alters the presence or concentration of a detected biomarker which has a direct relation to the risk of suffering from renal damage, the bioindicator serves as an informer to modify any treatment or exposure to any nephrotoxic agent.

A preferred embodiment of the use of a protein or any fragment thereof is the use wherein the renal damage or progress of the renal damage is due to the administration of at least one nephrotoxic agent. According to the more preferred embodiment of the use of the protein or any fragment thereof, the nephrotoxic agent is an aminoglycoside antibiotic. Another even more preferred embodiment is the use wherein the aminoglycoside antibiotic is gentamicin.

According to the more preferred embodiment of the use of the protein or any fragment thereof, the nephrotoxic agent is cisplatin.

Another aspect of the present invention relates to a kit comprising, at least, one or more probes capable of recognising at least one protein, or any combination thereof, selected from the list comprising

a protein having at least 60% identity in respect of amino acid sequence SEQ ID NO: 1 or SEQ ID NO: 2, or any fragments thereof,

a protein having at least 80% identity in respect of amino acid sequence SEQ ID NO: 3 to 11, or any fragments thereof, or

a protein having at least 90% identity in respect of amino acid sequence SEQ ID NO: 12 to 14, or any fragments thereof.

A preferred embodiment relates to the kit, wherein the probes recognise at least one protein, or any combination thereof, selected from the list comprising SEQ ID NO: 1 to 14, or any fragments thereof.

Another preferred embodiment relates to the kit, wherein the probe/s recognise/s a protein having at least 60% identity in respect of SEQ ID NO: 1 and/or a protein having at least 60% identity in respect of SEQ ID NO: 2, or any fragments thereof. According to a more preferred embodiment, the probe/s recognise/s the protein SEQ ID NO: 1 and/or SEQ ID NO: 2, or any fragments thereof.

Another preferred embodiment of the present invention refers to the kit, wherein the probes recognise a protein at least 60% identical to SEQ ID NO: 1, or a fragment thereof. Preferably the probes recognise SEQ ID NO: 1, or a fragment thereof.

Another preferred embodiment of the present invention refers to the kit, wherein the probes recognise a protein at least 80% identical to SEQ ID NO: 5, or a fragment thereof. Preferably the probes recognise SEQ ID NO: 5, or a fragment thereof.

Another preferred embodiment of the present invention refers to the kit, wherein the probes are attached to a solid support.

A probe is a substance that is usually (but not necessarily) marked and that is used to detect, identify and/or quantify any protein of the present invention or any fragment thereof. The probe may be, but without limitation, a probe that reacts with thiol, biotin, avidin, streptavidin or peptin groups. The solid support is preferably a gel, for example, but without limitation, an agarose gel or polyacrylamide gel.

Another preferred embodiment relates to a kit wherein the probes are antibodies to recognise the protein, or any fragments thereof. Said antibodies may be polyclonal or monoclonal.

Another aspect of the present invention relates to the use of the kit described in precedent paragraphs, for determining a renal damage, or for predict the progression of a renal damage, in an isolated sample from an individual.

Another aspect of the present invention relates to the use of the kit described in precedent paragraphs for determining the risk of developing the renal damage, or for determining a renal damage, or for predicting the progression of a renal damage, in an isolated sample from an individual.

Throughout this description and the claims, the word “comprises” and its variants do not intend to exclude other technical characteristics, additives, components or steps. For experts in the art, other objects, benefits and characteristics of the invention will be inferred partly from the description and partly from the practice of the invention. The following drawings and examples are provided by way of illustration and are not intended to limit the present invention in any way.

### BRIEF DESCRIPTION OF THE FIGURES

FIG. 1. Shows the survival rate and appearance of renal damage markers in rats treated with gentamicin.

A. Survival rate shown as a percentage of the animals surviving in each group after 7 days treatment. B. Representative images of Western blot analysis of kidney injury 1 molecule (KIM-1) and bone morphogenetic protein 7 (BMP-7) expression levels in kidney homogenates from 3 randomly selected control and 3 gentamicin rats after 7 days treatment.

FIG. 2. Shows representative images of the cortex and papilla of renal tissue sections from rats treated with gentamicin.

Representative images of the cortex (A-D) and papilla (E-H) of renal tissue sections stained with hematoxilin and eosin, from control rats (n=3) and gentamicin treated rats (n=3), randomly selected. Images A, B, E and F were taken at 400× magnification, while images C, D, G and H were taken at 1000× magnification.

FIG. 3. Shows the SDS-PAGE separation of urinary proteins from control rats and rats treated with gentamicin.

The proteins were separated by SDS-PAGE in polyacrylamide gels at 6% (A) and 15% (B), subsequently stained with Coomassie Brilliant Blue. The proteins present in bands (1-24) were determined by LC-ESI-Q-TOF and listed in table 3.

Lane 1: molecular weight patterns. Lane 2: Control urine. Lane 3: gentamicin group urine.

FIG. 4. Shows two-dimensional (2D) electrophoresis images of differentially expressed proteins.

The quantitative intensity analysis used in the Image Master 2D Platinum software (GE Healthcare) revealed 129 differently expressed proteins between the normal and gentamicin urine samples. The spots were marked with the numbers corresponding to those in table 4 and identified by means of LC-ESI-Q-TOF and MALDI-TOF. Each one of the gels shown in this figure is representative of 8 gels obtained with urine from animals randomly selected from each group, each one analysed in duplicate.

FIG. 5. Shows the urinary proteomics. 2D gel images of differentially expressed reg IIIb (A) and gelsolin (B).

Spots were subject to quantitative intensity analysis, labelled with numbers corresponding to those in table and identified by LC-ESI-Q-TOF mass spectrometry. Each gel shown in this figure is representative of 8 gels obtained with urine from 4 randomly selected animals in each group, each one analyzed in duplicate. reg IIIb protein is the spot 1; P25031 accession number, MW 20 kDa, PI 7.56. Gelsolin is the spots 2,3 y 4, Q68FP1 accession number, MW 86.1 kDa, PI 5.75

FIG. 6. Shows western blot analysis of reg IIIb (A) and gelsolin (B), Serum creatinine and BUN concentration

A. Western blot in the urine of 6 randomly selected rats treated with vehicle (control), gentamicin or cisplatin. Arrows indicate the full length and t-gelsolin fragment.

B. Serum creatinine and BUN concentration of rats as in A. *, p<0.05 vs. control; •p<0.05 vs. gentamicin.

FIG. 7. Shows time course evolution of urinary reg IIIb (A) and gelsolin (B).

A. Representative images of Western blot analysis of urinary reg IIIb (A) and gelsolin (B), and NAG excretion and proteinuria of rats treated with gentamicin; Serum creatinine concentration; Western blot analysis of KIM-1, NGAL and PAI-1; Representative image of renal sections stained with hematoxilin and eosin from rats treated with gentamicin during 3 days. n=3 in all experiments. *, p<0.05 vs. time 0.

FIG. 8. Shows Western blot analysis of serum level of reg IIIb (A) and gelsolin (B), renal perfusion experiments and RT-PCR analysis.

Representative images of Western blot analysis of serum level of reg IIIb (A.1) and gelsolin (B.1) from 2 randomly selected rats treated with vehicle (control) or gentamicin during 6 days.

Renal perfusion experiments. Representative images of Western blot analysis of the urinary level of reg IIIb (A.2) and gelsolin (B.2) from rats treated with gentamicin during 6 days, and then subject to renal perfusion with Krebs solution, immediately before the beginning of perfusion and 1 and 2 hours during it; n=3.

A3. Renal tissue Reg IIIb (A.3), Gelsolin (B.3) and GAPDH gene expression by RT-PCR from 3 randomly selected rats treated with vehicle (control) or gentamicin during 3 and 6 days. C.

### EXAMPLES

### Example 1

### Materials and Methods

### 1.1. Animals and Experimental Protocol.

Female Wistar rats were used weighing 200-250 g. The animals were allocated under controlled environmental conditions in individual metabolic cages, for individual urine sample collection every 24 hours. Normal feed and water were administered ad libitum. Rats were randomly divided in two groups: (i) control group (C), receiving daily placebo i.p. during 6 days, and (ii) gentamicin group (G), receiving gentamicin i.p. (150 mg/kg of body weight) during 6 days. On day 7, the animals were anaesthetised with sodium pentobarbital and the kidneys were perfused by the aorta with saline (0.9% NaCl) to eliminate the blood. The kidneys were immediately dissected. One was frozen in liquid nitrogen and subsequently kept at −80° C. for Western blot studies, and the other was soaked in p-formaldehyde at 3.7% for histological studies. Blood samples were also obtained in heparinised capillaries at different time points by a small incision in the tail tip. Blood was centrifuged and serum was kept at −80° C. until use. Urine was cleared by centrifugation at 10,000 g and 4° C. during 10 minutes, and kept at −80° C. until use.

### 1.2. Histological Studies.

The kidneys were kept in p-formaldehyde all night at 4° C. Next, paraffin blocks were made and 5-μm tissue sections were cut and stained with hematoxilin and eosin. Photographs were taken under an Olympus BX51 microscope connected to an Olympus DP70 colour digital camera.

### 1.3. Biochemical Measurements.

Serum and urinary creatinine and urea nitrogen concentration were measured using commercial reactive strips (Roche Diagnostics) and the Reflotron® automated analyser (Roche Diagnostics). The creatinine concentration measurement had a lower detection limit of 0.5 mg/dL. Urine protein concentration was measured by the Bradford method (Bradford, 1976. Anal Biochem, 72: 248-254). Urine NAG content was indirectly determined by the level of NAG activity. NAG enzymatic activity was determined by a colorimetric method based on the conversion of 3-cresolsulfonphthaleinyl-N-acetyl-D-glucosaminide into the purple 3-cresol-cresolsulfonphthaleinyl, measured at 580 nm using a plate photometer (Thermo). To measure NAG activity a commercial kit was used (Roche Diagnostics) following the manufacturer's instructions.

### 1.4. Western Blot.

Western blots were run with (i) urine samples (21 μl per sample), (ii) tissue extracts (100 μg total protein per sample) prepared by homogenizing the kidneys with a tissue mixer (Ultra-Turrax T8, IKA®-Werwe) at 4° C. in homogenization buffer (140 mM NaCl, 20 mM Tris-HCl pH=7.5, 0.5 M ethylenediaminetetraacetic acid—EDTA-, 10% glycerol, 1% Igepal CA-630, 1 μg/mL aprotinin, 1 μg/mL leupeptin, 1 μg/mL pepstatin A, 1 mM phenylmethylsulphonyl fluoride—PMSF—), or (iii) albumin-free blood serum. Albumin was removed from serum with a column-based, commercial kit based on the immunological retention of rat albumin (Qproteome Murine Albumin Depletion Kit, Quiagen). Samples were separated by electrophoresis in 10-15% acrylamide gels (Mini Protean II system, BioRad). Immediately, proteins were electrically transferred to an Immobilon-P membrane (Millipore). Membranes were probed with antibodies against KIM-1 (R&D Systems), bone morphogenetic protein 7 (BMP-7, Santa Cruz Biotechnology), NGAL (MBL), PAI-1 (BD Biosciences), reg IIIb (R&D Systems), and gelsolin (Santa Cruz Biotechnology).

### 1.5. One-Dimensional and Two-Dimensional (1D and 2D) Electrophoretic Protein Separation.

Urine was concentrated and desalted by force filtration through centrifugation in Amicon Ultra columns with 5 K cut-off (Millipore). Protein concentration was determined by the Bradford method. For 1D electrophoretic separation 100 mg of total protein was made to run in 6 and 15% acrylamide gels (Mini Protean II system, BioRad, Madrid, Spain) and stained with Coomassie Brilliant Blue G-250. For two-dimensional electrophoresis (2D), urine proteins were precipitated with the Clean-Up kit (GE Healthcare) following the manufacturer's instructions. 100 mg of protein of each sample were rehydrated in 7 M urea, 2 M thiourea, 4% (w/v) Chaps, 0.5% ampholytes, pH 4-7 or 4.5-5.5), 50 mM dithiothreitol (DTT) and bromophenol blue, and isoelectrically focused (500-8,000 V) through 18-cm long immobilised pH gradient (IPG) strips, pH 4-7 or 4.5-5.5 (GE Healthcare, Madrid, Spain), using an IPGphor apparatus (GE Healthcare). The IPG strips were pre-equilibrated during 15 minutes in equilibration buffer [50 mM Tis-HCl, pH=8.8, 6 M urea, 30% (v/v) glycerol, 2% (w/v) sodium dodecylsulphate (SDS), 0.01% (w/v) bromophenol blue containing 1% (w/v) DTT, and another 15 minutes in equilibration buffer containing 2.5% (w/v) iodoacetamide. Then, IPG strips were transferred to 18-cm long 12% acrylamide gels and separated by electrophoresis with a SE 600 Ruby apparatus (GE Healthcare). Gels were fixed overnight in 30% ethanol, 10% acetic acid and silver stained with a commercial kit (GE Healthcare). Unless otherwise indicated, all reagents were from Sigma.

For visualisation and analysis, stained gels were scanned (Image Sanner, GE Healthcare, Madrid, Spain), processed and statistically analysed with the Image Master 2D Platinum 6.0 software (GE Healthcare, Madrid, Spain). Spot discrimination was done with the following parameters: (i) smooth factor: 2; (ii) minimal area: 5 pixels; (iii) saliency: 100. Analysis was visually corrected for artefact elimination. For each individual spot, background was subtracted and individual intensity volume was normalised by total intensity volume (all-spot intensity). For comparison of the same spot among gels, a minimum of a two-fold difference was established to consider a differential expression.

### 1.6. Two-Dimensional Liquid Chromatography (Chromatofocus+Reverse Phase) (2D-LC).

A ProteomeLab PF2D system (Beckman Coulter) was used. Concentrated and desalted urines were diluted in optimised buffer (7.5 M urea, 2.5 M thiourea, 12.5% glycerol, 62.5 mM Tris-HCl, pH=7.8-8.2, 2.5% (w/v) n-octylglucoside, 6.25 mM tris (carboxyethyl) phosphine hydrochloride) during 30 minutes at room temperature. This buffer was then replaced by start buffer (pH 8.5±0.1, Beckman Coulter) using a PD-10 column (GE Healthcare). The first fraction of 3.5 ml was collected and protein concentration was quantified. Next, 1 mg of each sample was placed in the chromatofocus ion exchange column and eluted into fractions of pH 0.3 according to the isoelectric point by means of linear descending pH gradient (8.5 to 4). Each pH fraction was then separated at 50° C. according to protein hydrophobicity by reverse phase high performance liquid chromatography using a C18 non-porous column (RP-HPLC) implemented with a linear gradient of 5-100%, 0.08% TFA in acetonitrile at 0.75 mL/min where A is 0.1% TFA in water. The reverse phase fractions of 0.6 min were collected and kept at −80° C. Absorbance was measured at 214 nm in the second dimension, and data were analysed using the 32 Karat and Delta View software (Beckman Coulter).

1.7. Protein Identification from 1D, 2D and 2D-LC Separations.

The bands and spots of interest from the 1D and 2D separations (respectively) were cut off the gels. Each gel cut-out was dehydrated in acetonitrile, vacuum evaporated and resuspended in NH₄HCO₃. 2D-LC fractions were also vacuum evaporated and residues resuspended in NH₄HCO₃. Thus, the samples from 1D, 2D and 2D-LC were treated identically. Samples were then reduced with 10 mM DTT in 50 mM NH₄HCO₃ at 56° C., and alkylated with iodoacetamide in 50 mM NH₄HCO₃. Next, proteins were in-gel digested with porcine trypsin (Promega) during 30 minutes at 4° C. Peptides were then extracted with 0.5% (v/v) trifluoroacetic acid (TFA). The solution was vacuum evaporated and peptides were dissolved in 0.1% (v/v) formic acid under sonication. Peptide-containing solutions were injected in a LC9 ESI-QUAD-TOF mass spectrophotometer QSTAR XL (Applied Biosystems) with a 1100 micro HPLC (Agilent). A wide pore 150×0.32 mm (5 m) Supelco column (Discovery B10) was used with a flow of 7 L/min. MS/MS spectra were obtained. Protein identification was performed with the MASCOT software (www.matrixscience.com) against non redundant protein sequence databases (Swiss Prot and NCBI). Mass tolerance was set at 50 ppm, MS/MS tolerance was 0.5 Da, and the taxonomic status was Rattus. Only significant hits, as identified by MASCOT probability analysis, were considered and at least one peptide watch with ion score above 20 was set as the threshold of acceptance. In some cases, where the LC-ESIQUAD-TOF procedure did not produce unambiguous protein identification, or in randomly selected spots, for confirmation, proteins were identified with an Ultraflex I MALDI-TOF mass spectrophotometer (Bruker Daltonics) at the Proteomic Service of the Universidad de Salamanca-CSIC Cancer Research Centre (Salamanca, Spain).

### 1.8 Protein Identification by Mass Spectrometry

The spots of interest from 2D separations were cut off the gels, dehydrated in acetonitrile, vacuum-evaporated and resuspended in NH₄HCO₃. 2D-LC fractions were also vacuum-evaporated and residues ressuspended in NH₄HCO₃. Samples were then reduced with 10 mM DTT in 50 mM NH₄HCO₃ at 56° C., and alkylated with iodoacetamide in 50 mM NH₄HCO₃. Proteins were in-gel digested with porcine trypsin (Promega), and peptides were extracted with 0.5% (v/v) trifluoroacetic acid (TFA), vacuum-evaporated and redissolved in 0.1% (v/v) formic acid. Peptide-containing solutions were injected in a LC-ESI-QUAD-TOF mass spectrophotometer QSTAR XL (Applied Biosystems) with an 1100 micro HPLC (Agilent). A wide pore 150×0.32 mm (5 μm) Supelco column (Discovery B10) was used. MS/MS spectra were obtained. Protein identification was performed with the MASCOT software (www.matrixscience.com) against non redundant protein sequence databases (Swiss Prot and NCBI). Mass tolerance was set at 50 ppm, MS/MS tolerance was 0.5 Da, and the taxonomic status was Rattus. Only significant hits, as identified by MASCOT probability analysis, were considered and at least one peptide match with ion score above 20 was set as the threshold of acceptance. For confirmation, some proteins where also identified with an Ultraflex I MALDI-TOF mass spectrophotometer (Bruker Daltonics).

### 1.9. Gene Expression Analysis.

RT-PCR-amplification of reg IIIb, gelsolin and GAPDH was performed with the next primers: for rat reg IIIb, SEQ ID NO: 15 and SEQ ID NO: 16; for rat gelsolin, SEQ ID NO: 17 and SEQ ID NO: 18; for rat glyceraldehyde 3-phosphate dehydrogenase (GAPDH), SEQ ID NO: 19 and SEQ ID NO: 20. PCR conditions were: 1×(95° C.×4 min); 30×(95° C.×1 min+Tm×1 min); 1×72° C.×10 min); where Tm was 55.5° C. for reg IIIb, 55.0° C. for gelsolin, and 55.9° C. for GAPDH.

### 1.10. Renal Excretion Studies.

At the end of the treatment, rats treated with gentamicin during 6 days were anesthetized and an extracorporeal circuit for kidney perfusion was set up, as described elsewhere (69), with some modifications. Briefly, the renal artery, vein and ureter of the right kidney were ligated. The renal artery and vein of the left kidney and the urinary bladder were canulated. Oxygenated and warm (37° C.) Krebs-dextran [40 g/L of dextran (molecular weight 64K-76K) in Krebs solution (118.3 mM NaCl, 4.7 mM KCl, 1.8 mM CaCl2, 1.2 mM MgSO4, 1.2 mM KH2PO4, 25 mM NaHCO3, 0.026 mM EDTA, 11.1 glucose, pH=7.4)] was perfused through the renal artery at 3 mL/min, and was discarded through the renal vein. Urine fractions were collected from a catheter placed in the urinary bladder, starting before the perfusion with Krebs (when blood was still passing through the kidney), and during 2 hours after perfusion with Krebs started. All urine samples were kept at −80° C. until assayed by Western blot for the presence of reg IIIb and gelsolin.

### 1.11. Statistical Analysis.

Data are represented as the mean±standard error of n experiments performed, as indicated in each case. Except for the study of proteomic results (as indicated above), statistical comparisons were assessed by the one-way ANOVA analysis. Normality distribution was assessed (passing at least three of the following tests: normality of residuals by asymmetry, normality of residuals by kurtosis, normality of residuals by omnibus and equality of variance by modified Levene. The Scheffe test was used to determine the statistical significance. A value of p<0.05 was considered significant.

### Example 2

### Characterisation of Gentamicin-Induced Kidney Damage

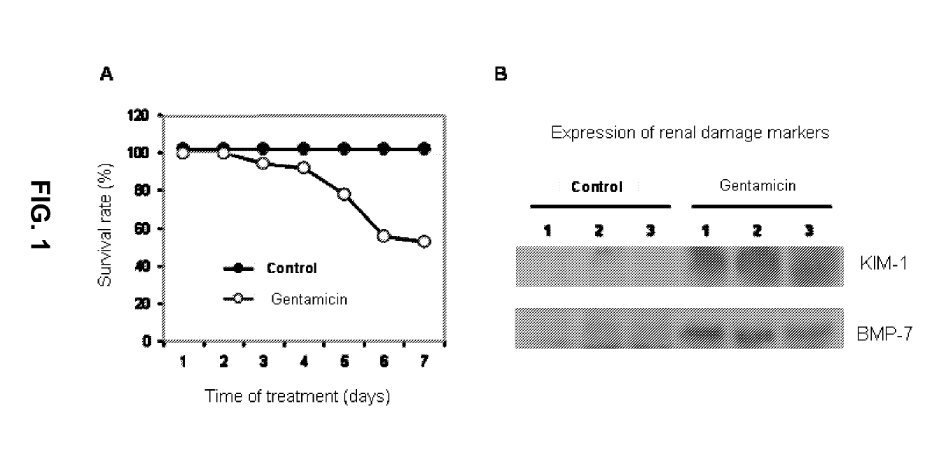

After 7 days treatment, gentamicin caused a marked renal damage (acute kidney injury or acute renal failure) with an associated mortality of about 50% (FIG. 1 and table 2). Surviving animals coursed with a small but significant weight loss and polyuria. Acute renal failure was further characterised by a dramatic increase in serum creatinine and BUN concentration, indicating a reduction of GFR. NAG excretion also increased over a large area, indicating extensive tubular damage. Proteinuria was also evident in the urine of animals treated with gentamicin (Table 2).

TABLE 2
Body weight (shown as a percentage of the initial weight), plasma creatinine concentration,
blood urea nitrogen (BUN), proteinuria, NAG excretion and urinary flow in control and
gentamicin rats after 7 days treatment at p < 0.05 in respect of the control group.
n = 12 at the beginning of the experiment in both groups.

| | Δ | | | | | Urine |
|---|---|---|---|---|---|---|
| | Weight | Creatinine | BUN | Proteinuria | NAG | flow |
| | (%) | (mg/dL) | (mg/dL) | (mg/day) | (UA/day) | (mL/day) |
| Control | 105 ± 4 | <0.5 | 45.9 ± 1.9 | 7.3 ± 0.9 | 54 ± 11 | 10 ± 1 |
| Gentamicin | 95 ± 3^{a} | 3.3 ± 0.4^{a} | 435.8 ± 47.9^{a} | 40 ± 7.4^{a} | 744 ± 212^{a} | 18 ± 3^{a} |

^{a}p ≦ 0.05 with respect to the control group.
n = 12 at the beginning of the experiment in both groups

### Example 3

### Renal Histopathological Study

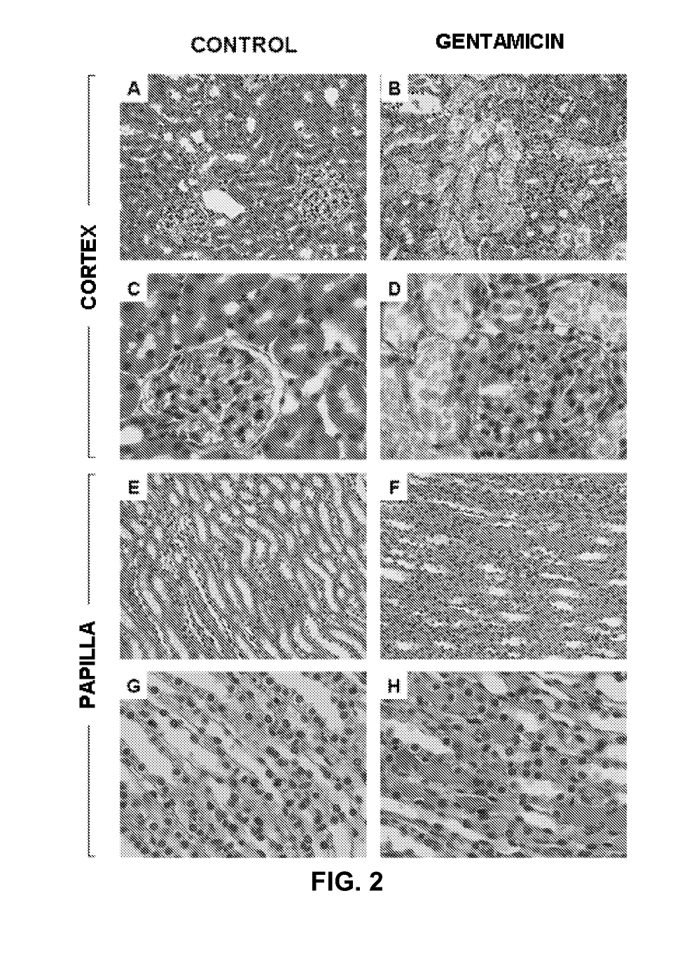

Kidney sections stained with hematoxilin-eosin (FIG. 2) revealed a clear tubular necrosis in gentamicin rats, where massive epithelial destruction can be observed. An important modification of the glomerules is not evident. On a papillary level, obstruction of the collector tubules with hyaline material is common in animals treated with gentamicin. These studies provide the morphological backup for at least part of the extensive renal dysfunction observed.

### Example 4

### Differential Proteomic Analysis of the Urine

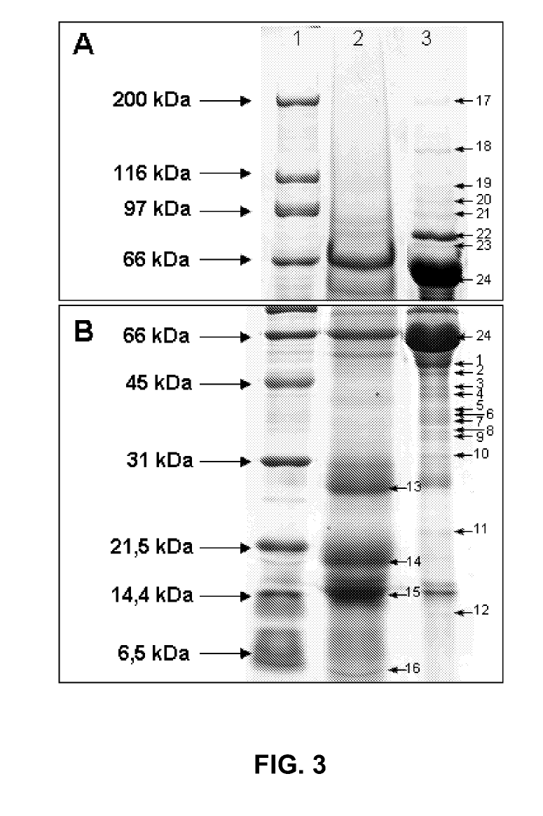

In this study, three different proteomic approaches (1D-SDS-PAGE, 2D and 2D-LC) and two mass spectrometry techniques (LC-ESI-QUAD-TOF and MALDI-TOF) were used for protein identification. This combination proved partially overlapping and partially non-redundant. The representative results of 1D are shown in the figures. FIG. 3 represents a typical SDS-PAGE profile of the urine from control and gentamicin rats, from which 24 differentially abundant bands were extracted and analysed, each one containing several proteins. Bands 13 to 16 were most abundant in the control group, whereas bands 1 to 12 and 17 to 24 were most abundant in the gentamicin group. The proteins identified by this approach are listed in table 5.

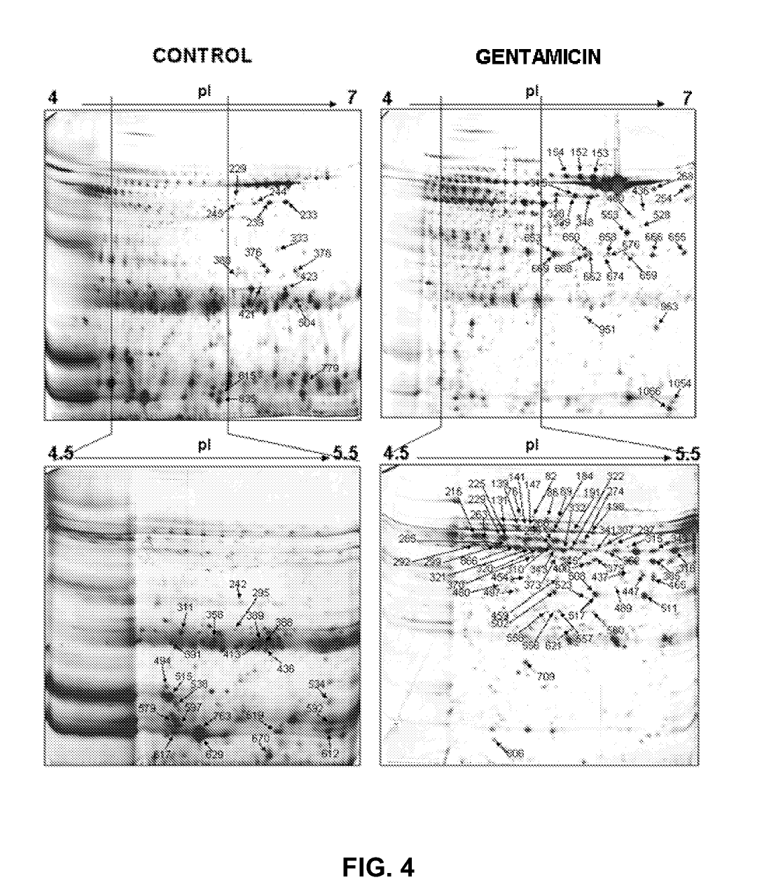

In pilot studies, the protein distribution profiles of control and gentamicin urines were determined according to their isoelectric point. Most proteins were included in the pH interval of 4 to 7. In pilot studies, the authors used IPG strips covering a wide pH range (2-10). Because there was a defect in the range beyond pH 7, and also because comparatively few proteins were lost between the pH range 7-10, the authors decided to perform the experiments in the 4 to 7 pH range for the first dimension of the 2D separation. The upper panels of FIG. 4 show a representative image of a 2D gel (pH range 4-7) of urine samples from control rats treated with gentamicin. Great similarity is observed between the samples from animals of the same group, and high reproducibility was obtained when the 2D separation was repeated using the same sample. Many proteins concentrated in the 4.5-5.5 pH range. For this reason, the same 2D separations of the same urine samples were also performed in this pH range. A significant image of this is shown in the lower panels of FIG. 4. In both cases, differentially present and statistically significant spots were recognised and numbered for chemical identification between control and gentamicin groups. (FIG. 4 indicates the numbers associated to the spots for the group in which the specific protein appears more abundantly).

The urine proteome of both groups was substantially different. In the first place, significantly more spots were identified in the urine of gentamicin treated rats than in control rats. In the 4-7 pH range, 606 spots from the control group were identified and 933 from the gentamicin group. In the 4.5-5.5 pH range, the numbers were 358 and 724, respectively. Of these, 129 differentially expressed spots were found between the groups (37 overexpressed in the gentamicin group and 92 in the control group). Of these 129 spots, the mass spectrometry data identified 98 of them in protein databases, corresponding to isoforms or variants modified post-transcription of 34 proteins (23 increased in the gentamicin group and 11 in the control group; see table 4).

A complementary study of the differential proteome in the urine of two control animals and two treated with gentamicin was performed following the 2D-LC procedure. The similarity between the samples of the same group was good and the reproducibility of the results of different experiments using the same sample was also very high. The MS analysis of the differentially abundant fractions identified 22 proteins (see table 5). Many of these proteins coincided with those found following the 2D procedure such as 2-HS-glycoprotein, hemopexin, serum albumin, T 1 kininogen, transthyretin, vitamin D binding protein, and urinary proteins 1, 2 and 3. Many of these proteins were also identified in several reactions, confirming the existence of variants or isoforms. Interestingly, this procedure identified several proteins undetected by 1D and 2D. This is the case for example of 2-microglobulin and cystatin C. One must mention that as a result of the liquid fractioning, a better coverage of the sequences was obtained for most of the proteins also identified following the 2D procedure.

The protein separation procedures used, specifically 1D, 2D and 2D-LC, proved to be partially overlapping but also partially non-redundant, making evident their complementary value.

### Analysis of the Results of Examples 1-4

In the present invention urine has been selected as the most suitable body sample for routine analyses on the scale of the population. In the case of renal illnesses, this is not only convenient because of its simple non-traumatic accessibility, but also due to its direct contact with the tissues in which the pathological events occur, from which it is easy to gather important diagnostic material.

Urine sampling can be carried out after 24 hours of starting treatment or administration of aminoglycoside antibiotics, against random urine samples. 24-hour sampling allows a finer and more precise normalisation through calculation of the daily excretion of any of the proteins of the invention (markers). However, its use in the clinical context is limited to a sub-set of patients. Random samples, on the other hand, serve better to analyse the population in general. However, the level of presence of the potential markers may strongly depend on the urine concentration, which can alter results and conclusions. Attempts to normalise urine concentration (i.e., according to urine creatinine concentration) can also lead to distorted results, since the parameters of normalisation are frequently altered in pathological conditions.

One attempt to resolve these inconveniences would be to act by creating urinary profiles of any of the proteins of the invention whose presence in the urine is altered (whether increased or decreased) in relative terms, as a fraction of all the proteins excreted as the result of an aggression (for example, a drug). For application in clinical practice, the magnitude of a marker protein in a specific diagnosis would be the result of its normalisation in respect of the total quantity of proteins in the analysed urine sample. Thus, with this objective, in the present study the authors have normalised urine samples according to the protein content using the same amount of proteins per sample, and next, studied the relative presence of any of the proteins of the invention, identified by differential proteomic techniques.

From a theoretical point of view, an increase or decrease in the absolute or relative presence of any of the proteins of the invention in urine may be the result of several factors: (i) alterations in the glomerular filtration and in the tubular secretion of proteins from blood or the extracellular renal space; (ii) markers may come from renal tissues damaged or in repair; (iii) the altered presence may also be the result of an impeded reabsorption capacity in general or at tubular level specifically; (iv) finally, and very importantly, the markers may come from synthesis, signalling pathways, transmembrane transport or exocytosis altered in existing renal tissues or unaltered or from specific compartments.

In the present invention a number of renal damage marker proteins have been found which appear increased in the urine of rats nephrointoxicated with gentamicin in respect of healthy (control) rats through the application of proteomic techniques (Table 6). It must be taken into account that the excretion of these marker proteins is underestimated because 14 rats of the gentamicin group showed an evident proteinuria. This means that the daily secretion of the marker proteins identified as increased in these animals is several times higher than their relative presence in urine (the excretion of proteins in the gentamicin group is more than 5 times higher than in the control group, Table 2). The analysis of the 1D and 2D electrophoretic profiles (FIGS. 3 and 4) of proteins in urine from both groups clearly reveals that the relative abundance of low molecular weight proteins (<35 KDa) is higher in the control rats. However, the proteins of medium molecular weight (MWW) (35-65 KDa) are relatively more abundant in gentamicin-treated rats. In the latter, from the 1D experiments it is particularly evident that the high molecular weight proteins (HMW) (65 KDa), absent in the rats from the control group, appear as a consequence of the treatment with gentamicin. These large proteins can be found with up to almost 200 KDa (FIG. 3, bands 17-22). The presence of HMW proteins in urine is normally considered a symptom of injury in the glomerular filtration barrier (GFB) which increases its permissiveness for larger proteins, normally excluded from ultrafiltration. However, high molecular weight proteins can also appear in urine as a result of the flaking off of renal structures in direct contact with the urine, such as those forming the filtration barrier, the different tubular segments, ureters or the bladder. A nephritic-type proteinuria resulting from a clear lesion in the glomerular filtration barrier is expected to produce the appearance of proteins with a non-specific range of molecular weights in urine. However, a more specific and subtle damage may also be considered that would only cause the appearance of some high molecular weight proteins in urine. It is noteworthy that in the case of animals treated with gentamicin of the present invention, most proteins appeared to concentrate around or below the upper size limit of the normally filtered proteins (for example, approximately the size of albumin), with relative few above said threshold.

However, together with these HMW proteins, the excretion of numerous low molecular weight (LMW) proteins also increases after treatment with gentamicin (see Table 6).

These data, as a whole, apparently indicate that this gentamicin regime, highly nephrotoxic and damaging for the kidney, only moderately altered the filtering properties of the glomerular filtration barrier, despite producing a marked renal insufficiency that is correlated to a mortality rate of 50% (FIGS. 1 and 2).).

The influence of tubular reabsorption and the secretion processes on the final composition of urine is not only a consequence of the effects of gentamicin on a tubular level. Also, the composition of the ultra-filtrate specifically alters protein reabsorption, presumably by processes of competition. Thus, specifically altered protein content in the ultra-filtrate as a result of this fact also specifically modifies its own tubular processing, additionally contributing a specific urinary marker of the aggression.

The results shown in FIGS. 1 and 2 point towards an evident tubular lesion produced by gentamicin, seriously reducing the recovery of proteins filtered on a tubular level (Table 2).

Table 6 mainly lists MMW and LMW proteins. As in many other renal diseases, most of these proteins come from blood and their urinary excretion appears increased after treatment with gentamicin, proving a general model of reduced tubular reabsorption.

Differentially-detected proteins (Table 6), common to other renal diseases include cathepsin B, some proteinases (alpha-1-antitrypsin, cystatin C, inter-alpha-trypsin inhibitor, serpin A3L, and various acute phase and immune response proteins (alpha-1-microglobulins, alpha-2-HS-glycoprotein, ceruloplasmin, complement C3 and C9, haptoglobin, hemopexin, immunoglobulins, lysozyme C, T kininogens). Their increased excretion could be explained by an increased presence in the ultra-filtrate (not as a result of a hyper-filtration, rather due to their higher levels in blood) combined with an imbalanced tubular reabsorption.

Of all the differentially detected proteins (Table 6), the increase in urinary values of the following proteins has a biomarker value with a diagnostic value of renal damage: protein Reg3B, fetuin B, Ras-related GTP-binding protein A, serpin A3L, subunit 1 of COP9, gamma subunit of ATP synthase, gelsolin, ribonuclease UK114, aminoacylase 1A, alpha-enolase, keratin-5, parvalbumin alpha, ribonuclease 4, serpin A3K. These proteins have not previously been related to obtaining useful data for diagnosing renal illnesses or used in the diagnosis itself. Except for gelsolin and serpin A3K, these proteins are normally absent from the blood. The increase of any of said proteins in blood is the result of the events occurring in the kidney provoked by gentamicin. Also, there are serious candidates for additionally exploring their use in the prognosis of the renal damage and/or acute renal failure produced by aminoglycoside antibiotics such as gentamicin.

In addition, a differential proteomic analysis of the kidney cortex revealed that gentamicin causes over-regulation of the proteins mainly related to (i) gluconeogenesis and glycolysis (for example, fructose 1,6-bisphosphatase and alpha-enolase); (ii) the transport and metabolism of fatty acids (for example, fatty acid transport protein, acetyl-CoA carboxylase, methylacyl-CoA racemase); (iii) the citric acid cycle (for example, ATP-specific succinyl CoA synthetase, malate dehydrogenase); and (iv) the stress response (for example, moesin, SPI 1, dnaK-type molecular chaperone). One exception is alpha-enolase, which is also increased in the urine of the test rats treated with gentamicin (Tables 4 and 6). This divergence in the modification of the protein model between renal tissue and urine probably reflects that most of the biomarker proteins of the present invention, derived from the kidney, are not the result of an increased production, but rather presumably of (i) an altered secretion from renal cells, or (ii) flaked or damaged tissues and cell residues excreted into urine.

TABLE 3
Proteins identified within bands in SDS-PAGE (one-dimensional
electrophoresis; 1D) in urine samples from rats treated with gentamicin in
respect of control rats, wherein proteins present in the gentamicin control
group are taken into account as well as those absent from the control, or
vice versa. Proteins also identified in two-dimensional electrophoresis
(2D) are highlighted in bold.

| | | Access | | | Cover |
|---|---|---|---|---|---|
| Band | Protein | No. | Score | Peptides | (%) |
| 1 | Vitamin D-binding protein | P04276 | 340 | 12 | 19 |
| | Alpha-1-antiproteinase | P17475 | 216 | 13 | 19 |
| | precursor | | | | |
| | Serum albumin precursor | P02770 | 156 | 7 | 9 |
| 2 | Serum albumin precursor | P02770 | 154 | 11 | 17 |
| | Ig gamma-2A chain C | P20760 | 69 | 2 | 6 |
| | Alpha-1-antiproteinase | P17475 | 37 | 1 | 1 |
| | precursor | | | | |
| | Gelsolin precursor | Q68FP1 | 21 | 2 | 2 |
| 3 | Serum albumin precursor | P02770 | 134 | 10 | 14 |
| 4 | Serum albumin precursor | P02770 | 27 | 1 | 2 |
| 5 | Haptoglobin precursor | P06866 | 52 | 5 | 13 |
| 6 | Haptoglobin precursor | P06866 | 165 | 9 | 23 |
| 7 | Serum albumin precursor | P02770 | 34 | 1 | 1 |
| | Inter-alpha-trypsin inhibitor | Q63416 | 25 | 1 | 0 |
| | heavy chain H3 precursor | | | | |
| 8 | Serum albumin precursor | P02770 | 428 | 19 | 25 |
| | C3 complement precursor | P01026 | 119 | 7 | 2 |
| | Hemopexin precursor | P20059 | 71 | 4 | 7 |
| | AMBP protein precursor | Q64240 | 41 | 2 | 3 |
| | Serpin A3L precursor | P05544 | 40 | 3 | 6 |
| 9 | C3 complement precursor | P01026 | 226 | 9 | 3 |
| | Serum albumin precursor | P02770 | 83 | 8 | 10 |
| | Serotransferrin precursor | P12346 | 43 | 1 | 1 |
| | AMBP protein precursor | Q64240 | 36 | 1 | 2 |
| 10 | Serum albumin precursor | P02770 | 139 | 8 | 11 |
| | Allele A Ig kappa chain C | P01836 | 36 | 1 | 7 |
| 11 | Plasma retinol-binding protein | P04916 | 23 | 1 | 4 |
| | precursor | | | | |
| 12 | Ribonuclease UK114 | P52759 | 31 | 1 | 7 |
| 13 | Allele B Ig kappa chain C | P01835 | 313 | 6 | 41 |
| 14 | Urinary protein 2 precursor | P81828 | 117 | 2 | 13 |
| | Allele A Ig kappa chain C | P01836 | 77 | 1 | 13 |
| | Ephrin type-A receptor 7 | P54759 | 39 | 1 | 12 |
| | precursor | | | | |
| | Glandular kallikrein-7 | P36373 | 23 | 1 | 3 |
| | precursor | | | | |
| 15 | Urinary protein 1 precursor | P81827 | 298 | 7 | 28 |
| | Prolactin-inducible precursor | O70417 | 82 | 3 | 21 |
| | Major urinary protein | P02761 | 48 | 1 | 4 |
| | precursor | | | | |
| | Urinary protein 2 precursor | P81828 | 37 | 1 | 13 |
| 16 | Not determined | | | | |
| 17 | Not determined | | | | |
| 18 | Ceruloplasmin precursor | P13635 | 74 | 4 | 3 |
| 19 | Not determined | | | | |
| 20 | Not determined | | | | |
| 21 | Not determined | | | | |
| 22 | Serotransferrin precursor | P12346 | 786 | 29 | 35 |
| | Ras-Related GTP-binding | Q63486 | 32 | 1 | 1 |
| | protein A | | | | |
| | COP9 signalosome, subunit 1 | P97834 | 32 | 1 | 1 |
| 23 | Serum albumin precursor | P02770 | 97 | 5 | 7 |
| 24 | Serum albumin precursor | P02770 | 806 | 28 | 41 |
| | T-kininogen 1 precursor | P01048 | 57 | 1 | 2 |
| | Hemopexin precursor | P20059 | 41 | 1 | 1 |

TABLE 4
Proteins with increasing or decreasing urine levels in the gentamicin group in respect
of the control group, identified by two-dimensional chromatography (2D).

| | | | | | | |
|---|---|---|---|---|---|---|
| | Access | Size MW | | | | |
| Protein | No. | (kDa)/pl | Spot No. | Score | Peptides | Cover (%) |

I. Proteins with increased expression levels in respect of the control.

| | | | | | | |
|---|---|---|---|---|---|---|
| Alpha-1-antiproteinase | P17475 | 46.1/5.7 | 292 | 259 | 7 | 14 |
| precursor | | | 299 | 334 | 11 | 22 |
| | | | 320 | 45 | 3 | 6 |
| | | | 332 | 363 | 11 | 26 |
| | | | 866 | 240 | 12 | 19 |
| Alpha-2-HS-glycoprotein | P24090 | 38/6.05 | 216 | 154 | 4 | 11 |
| precursor | | | 225 | 125 | 6 | 23 |
| | | | 229 | 148 | 4 | 11 |
| | | | 241 | 106 | 4 | 11 |
| | | | 248 | 109 | 4 | 11 |
| | | | 268 | 25 | 1 | 1 |
| | | | 274 | 134 | 4 | 14 |
| Serum albumin precursor | P02770 | 68.7/6.09 | 385 | 212 | 6 | 12 |
| | | | 650 | 106 | 3 | 4 |
| | | | 658 | 119 | 4 | 7 |
| | | | 659* | 318 | 23 | 40 |
| | | | 662 | 91 | 4 | 7 |
| Angiotensinogen precursor | P01015 | 52/5.37 | 310 | 146 | 4 | 8 |
| | | | 321 | 51 | 2 | 3 |
| | | | 322 | 260 | 7 | 16 |
| | | | 341 | 63 | 2 | 3 |
| | | | 352 | 135 | 4 | 9 |
| Haptoglobin precursor | P06866 | 38.6/6.1 | 497 | 220 | 12 | 26 |
| | | | 502 | 279 | 10 | 20 |
| | | | 508 | 230 | 10 | 26 |
| | | | 511 | 457 | 15 | 30 |
| | | | 517 | 266 | 11 | 26 |
| | | | 653 | 290 | 12 | 24 |
| | | | 668 | 226 | 11 | 23 |
| | | | 669 | 513 | 18 | 33 |
| Cathepsin B precursor | P00787 | 37.5/5.36 | 459 | 35 | 2 | 5 |
| Complement C3 precursor | P01026 | 186.5/6.12 | 447 | 132 | 6 | 4 |
| | | | 556 | 80 | 5 | 1 |
| | | | 557 | 145 | 7 | 3 |
| | | | 560 | 98 | 5 | 2 |
| Gelsolin precursor | Q68FP1 | 86.1/5.75 | 400 | 100 | 3 | 3 |
| | | | 403 | 85 | 3 | 3 |
| | | | 409 | 77 | 3 | 3 |
| Keratin | Q6P6Q2 | 62/7.61 | 621 | 64 | 1 | 2 |
| T-kininogen 1 precursor | P01048 | 48/6.08 | 184 | 253 | 8 | 17 |
| | | | 191 | 319 | 8 | 16 |
| | | | 198 | 442 | 11 | 30 |
| Serpin A3L | gi□29293811 | 46.5/6.04 | 379 | 337 | 9 | 22 |
| Vitamin D-binding protein | P04276 | 53.5/5.65 | 307 | 55 | 2 | 3 |
| | | | 316 | 555 | 16 | 31 |
| | | | 349 | 378 | 12 | 15 |
| Beta-2-glycoprotein 1 precursor | P26644 | 33.2/8.59 | 254 | 108 | 4 | 11 |
| | | | 268 | 178 | 3 | 11 |
| Alpha-enolase | P04764 | 47.1/6.16 | 436 | 79 | 5 | 10 |
| Fetuin-B precursor | Q9QX79 | 41.5/6.71 | 315 | 61 | 3 | 5 |
| | | | 328 | 247 | 7 | 7 |
| | | | 339 | 272 | 9 | 15 |
| | | | 348 | 288 | 9 | 30 |
| Hemopexin precursor | P20059 | 51.4/7.58 | 152 | 329 | 14 | 30 |
| | | | 153 | 375 | 14 | 29 |
| | | | 154 | 311 | 14 | 22 |
| Inter-alpha-trypsin inhibitor | Q63416 | 99.1/5.85 | 655 | 107 | 2 | 1 |
| heavy chain H3 precursor | | | 666 | 97 | 2 | 1 |
| Plasma retinol-binding protein | P04916 | 23.2/5.69 | 963 | 184 | 6 | 19 |
| precursor | | | | | | |
| Transthyretin precursor | P02767 | 15.7/5.77 | 1.066 | 38 | 3 | 19 |
| Gamma subunit of | P35435 | 30.2/8.9 | 951* | 94 | 7 | 26 |
| mitochondrial ATP-synthase | | | | | | |
| Reg3B | P25031 | 20.0/7.56 | 1054* | 107 | 7 | 46 |
| Aminoacylase-1A | Q6AYS7 | 46.0/6.03 | 528* | 433 | 26 | 75 |

II. Proteins with decreased expression levels in respect of the control.

| | | | | | | |
|---|---|---|---|---|---|---|
| Gelsolin precursor | Q68FP1 | 186.5/6.12 | 190 | 186 | 9 | 9 |
| Allele B Ig kappa chain C | P01835 | 11.6/4.97 | 311 | 126 | 5 | 49 |
| | | | 352 | 86 | 1 | 12 |
| | | | 387 | 107 | 2 | 13 |
| | | | 504 | 66 | 1 | 12 |
| | | | 518 | 36 | 1 | 12 |
| | | | 670 | 164 | 4 | 33 |
| | | | 391 | 113 | 2 | 13 |
| | | | 403 | 108 | 2 | 27 |
| | | | 413 | 127 | 4 | 41 |
| | | | 436 | 68 | 1 | 12 |
| Glandular kallikrein-7 precursor | P36373 | 28/5.63 | 376 | 54 | 3 | 9 |
| | | | 378 | 53 | 2 | 6 |
| | | | 494 | 59 | 1 | 3 |
| Keratin, type I cytoskeletal 15 | Q6IFV3 | | 675 | 80 | 3 | 5 |
| | | | 192 | 108 | 3 | 5 |
| | | | 187 | 109 | 3 | 5 |
| Urinary protein 1 precursor | P81827 | 11/6.67 | 597 | 60 | 1 | 10 |
| | | | 617 | 74 | 1 | 10 |
| | | | 619 | 72 | 1 | 10 |
| | | | 629 | 148 | 3 | 28 |
| | | | 835 | 65 | 1 | 10 |
| Urinary protein 3 precursor | P83121 | 11/6.85 | 597 | 67 | 1 | 13 |
| Pancreatic alpha-amylase | P00689 | 57.2/8.34 | 239 | 57 | 3 | 2 |
| precursor | | | 244 | 27 | 2 | 2 |
| Urokinase-type plasminogen | P29598 | 47/8.07 | 421 | 132 | 6 | 14 |
| activator precursor | | | | | | |
| Major urinary protein precursor | P02761 | 20.7/5.85 | 515 | 40 | 1 | 4 |
| | | | 570 | 136 | 5 | 18 |
| | | | 793 | 100 | 6 | 20 |

TABLE 5
Proteins with increasing or decreasing urine levels in the gentamicin
group in respect of the control group, identified by two-dimensional
liquid chromatography (2D-LC).

| | | |
|---|---|---|
| | Access | Fraction |
| Protein | No. | No. |

I. Proteins with increased expression levels in respect of the control

| | | |
|---|---|---|
| Beta-2-microglobulin precursor - Rattus norvegicus | P07151 | 1 |
| (Rat) | | |
| Alpha-2-HS-glycoprotein precursor - | P24090 | 4 |
| Rattus norvegicus (Rat) | | |
| AMBP protein precursor - Rattus norvegicus (Rat) | Q64240 | 1 |
| Complement component C9 precursor - | Q62930 | 1 |
| Rattus norvegicus (Rat) | | |
| Cystatin-C precursor - Rattus norvegicus (Rat) | P14841 | 3 |
| Haemoglobin subunit beta-1 - Rattus norvegicus (Rat) | P02091 | 1 |
| Hemopexin precursor - Rattus norvegicus (Rat) | P20059 | 5 |
| Allele A Ig kappa chain C - Rattus norvegicus (Rat) | P01836 | 2 |
| Lysozyme C type 1 precursor - Rattus norvegicus (Rat) | P00697 | 3 |
| Parvalbumin-alpha - Rattus norvegicus (Rat) | P02625 | 1 |
| Ribonuclease 4 precursor - Rattus norvegicus (Rat) | O55004 | 1 |
| Serpin A3K precursor - Rattus norvegicus (Rat) | P05545 | 4 |
| Serotransferrin precursor - Rattus norvegicus (Rat) | P12346 | 6 |
| Serum albumin precursor - Rattus norvegicus (Rat) | P02770 | 18 |
| T-kininogen 1 precursor - Rattus norvegicus (Rat) | P01048 | 2 |
| T-kininogen 2 precursor - Rattus norvegicus (Rat) | P08932 | 1 |
| Transthyretin precursor - Rattus norvegicus (Rat) | P02767 | 2 |
| Vitamin D-binding protein precursor - | P04276 | 3 |
| Rattus norvegicus (Rat) | | |

II. Proteins with decreased expression levels in respect of the control.

| | | |
|---|---|---|
| Protein-arginine deiminase type-2 - | P20717 | 2 |
| Rattus norvegicus (Rat) | | |
| Urinary protein 1 precursor - Rattus norvegicus (Rat) | P81827 | 10 |
| Urinary protein 2 precursor - Rattus norvegicus (Rat) | P81828 | 14 |
| Urinary protein 3 precursor - Rattus norvegicus (Rat) | P83121 | 2 |

TABLE 6
Summary of the proteins detected in urine with increased excretion in rats
treated with gentamicin in respect of control rats, through 1D, 2D and/or
2D-LC electrophoretic separations. Proteins highlighted in bold are those
not previously related to renal damage. Proteins are listed in alphabetical
order.

| Protein | Detection technique |
|---|---|
| Aminoacylase 1 | 2D |
| Albumin | 1D, 2D, 2D-LC |
| Alpha-enolase | 1D, 2D |
| Alpha-1-microglobulin | 1D, 2D-LC |
| Alpha-2-HS-glycoprotein | 1D, 2D-LC |
| Angiotensinogen | 2D |
| ATP synthase, gamma subunit | 2D |
| Beta-2-glycoprotein 1 | 2D |
| Beta-2-microglobulin | 2D-LC |
| Cathepsin B | 2D |
| Ceruloplasmin | 1D |
| Complement C3 | 1D, 2D |
| Complement C9 | 2D-LC |
| COP9 signalosome, subunit 1 | 1D |
| Cystatin C | 2D-LC |
| Fetuin B | 2D |
| Gelsolin | 1D, 2D |
| Haptoglobin | 1D, 2D |
| Haemoglobin, subunit beta 1 | 2D-LC |
| Hemopexin | 1D, 2D, 2D-LC |
| Ig gamma-2A, C chain | 1D |
| Ig kappa chain, C region | 1D, 2D-LC |
| Inter-alpha-trypsin inhibitor, heavy chain H3 | 1D, 2D |
| Inter-alpha-trypsin inhibitor, light chain | 1D, 2D-LC |
| Keratin 5 | 2D |
| Lysozyme C type 1 | 2D-LC |
| Parvalbumin alpha | 2D-LC |
| Ras-Related GTP-binding protein A | 1D |
| Reg3B | 2D |
| Retinol-binding protein | 1D, 2D |
| Ribonuclease 4 | 2D-LC |
| Ribonuclease UK114 | 1D |
| Serine (or cysteine) proteinase (inhibitor), clade F, | 2D |
| member 1 | |
| Serpin A3K | 2D-LC |
| Serpin A3L | 1D |
| Serotransferrin | 1D, 2D-LC |
| T-kininogen 1 | 1D, 2D, 2D-LC |
| T-kininogen 2 | 2D-LC |
| Transthyretin | 2D, 2D-LC |
| Vitamin D-binding protein | 1D, 2D, 2D-LC |

### Example 5

### Differential Proteomic Analysis of the Urine. Reg IIIb and Gelsolin

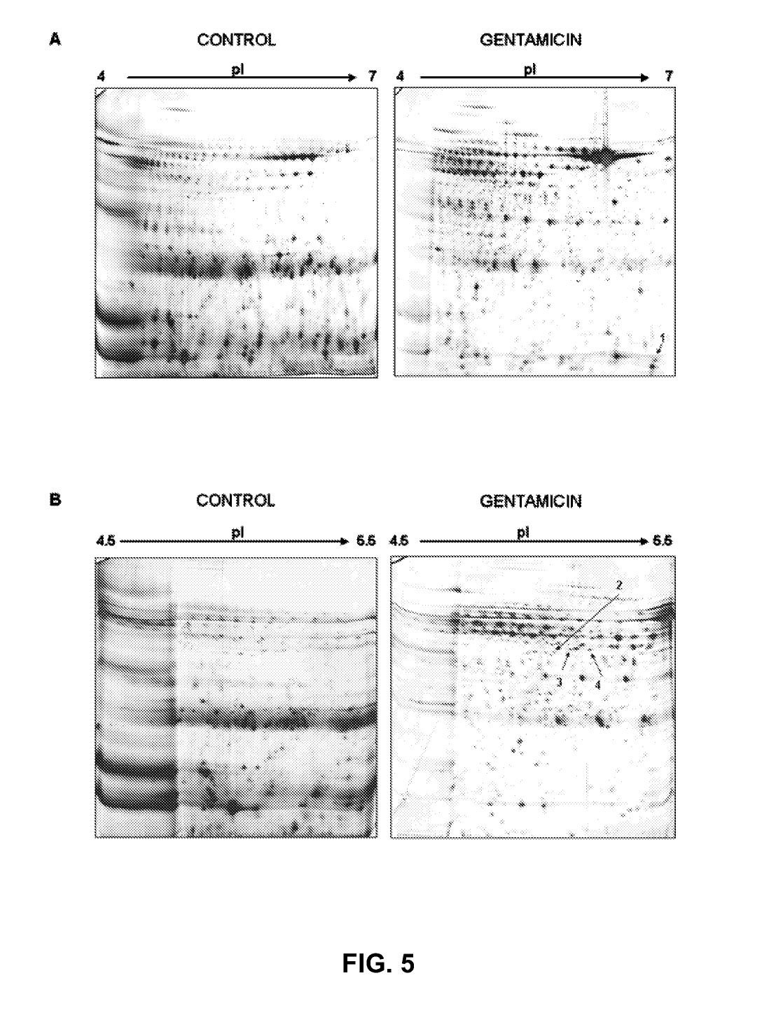

A representative image of 2D gels (pH range 4-7) of urines from control and gentamicin-treated rats is shown in the upper panels of FIG. 5. Many proteins concentrate in the rage of pH 4.5-5.5. For that reason, 2D separations in this pH range were also done with the same urines. A representative image of these latter is shown in the lower panels of FIG. 5. A great similarity was observed between samples from animals in the same group, and high reproducibility was obtained when repeating the 2D separation with the same sample, for quality assurance. However, the urine proteome of both groups is substantially different. Statistically significant, differentially present spots between control and gentamicin groups were recognized and numbered for chemical identification. Mass spectrometric analysis revealed the identity of three proteins increased in the urine of gentamicin-treated rats, which showed potential interest after discarding most of the other proteins, normally found in different proteinuric conditions. They were identified as regenerating islet-derived protein 3 beta (reg IIIb) and gelsolin (FIG. 5).

### Example 6

### Reg IIIb and Gelsolin are Differentially Excreted in the Urine of Rats Treated with Gentamicin

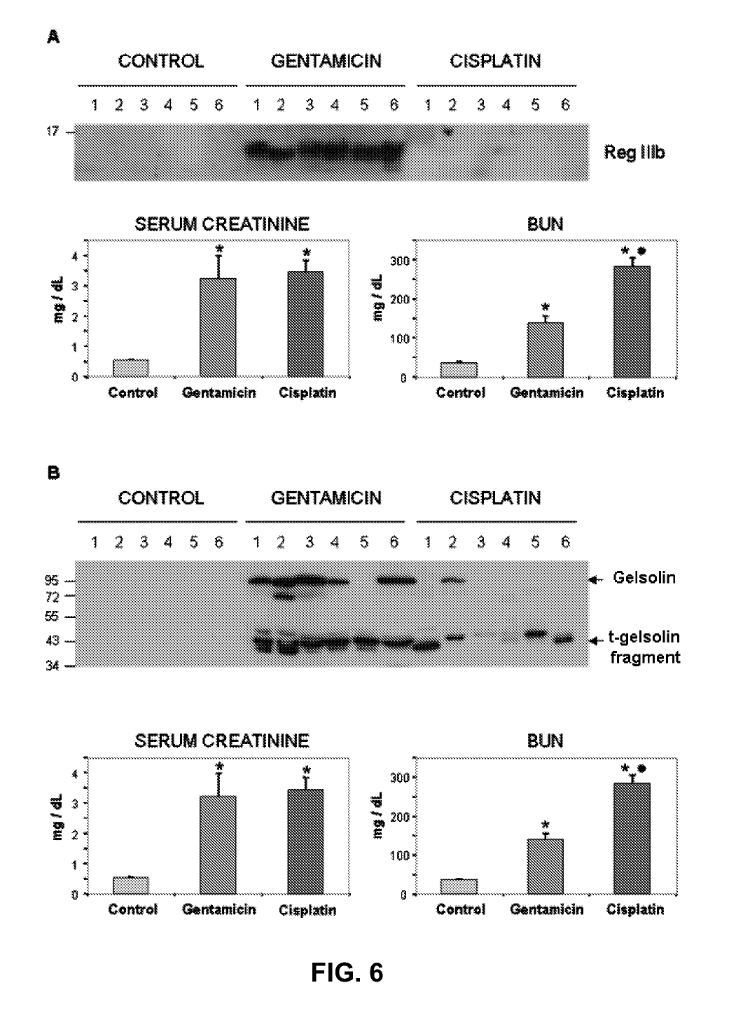

The increased urinary level of these proteins in the urine of gentamicin-treated rats was confirmed by Western blot analysis. Moreover, the urine from rats treated with a nephrotoxic regime of cisplatin was also analyzed. FIG. 6-B shows data on serum creatinine concentration and BUN from 6 control rats, 6 rats treated with gentamicin and 6 rats treated with cisplatin. It demonstrates that treated animals developed an overt renal failure. Those urines were also analyzed for their content in reg IIIb and gelsolin. FIG. 6-A clearly shows that the urinary level of reg IIIb is markedly increased only in gentamicin-treated animals, despite undergoing a similar degree of renal damage than cisplatin-treated rats. Western blot of gelsolin revealed two reactive bands. The higher one corresponds to the full length protein, whereas the lower one corresponds to a fragment thereof. The presence of gelsolin within the reactive bands was further re-confirmed by MS/MS mass spectrometry. Treatment with gentamicin induces the appearance in the urine of both the full length gelsolin and the ˜43 kDa fragment. However, the full length band was absent in the urine from all of the rats treated with cisplatin, except for one of them. Extensive analysis of the urine from other cisplatin-treated rats shows no presence of the full length band.

### Example 7

### Time Course Evolution of Reg IIIb and Gelsolin Urinary Excretion

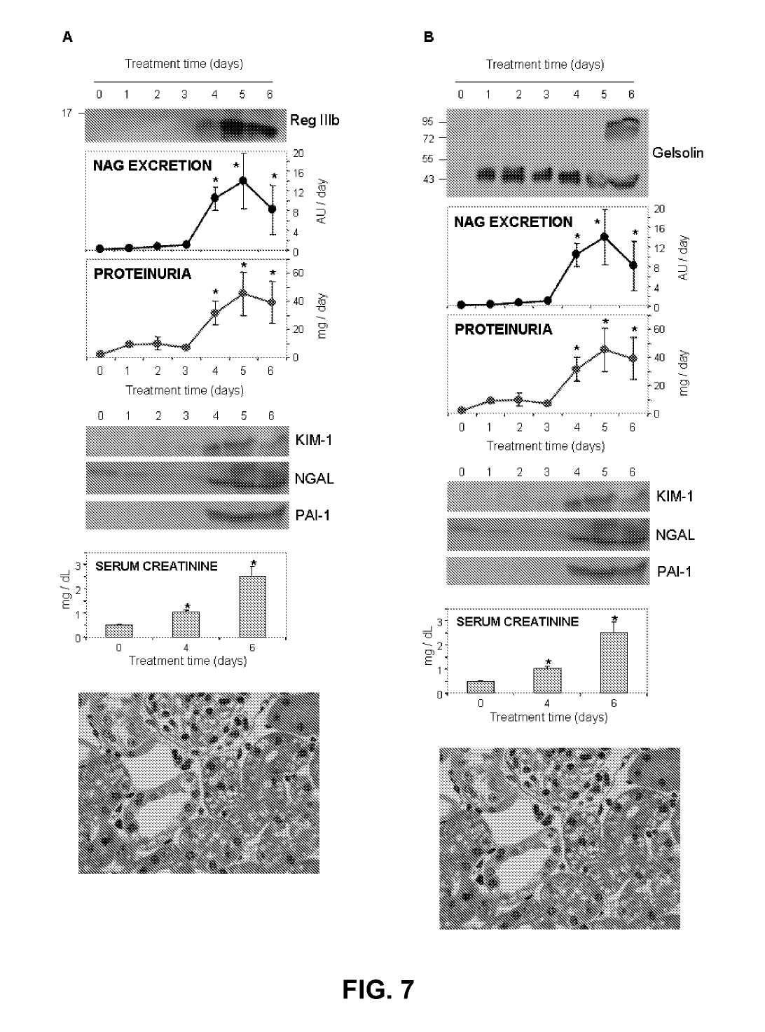

We further analyzed the time course evolution of the urinary excretion of these proteins in rats treated with gentamicin. FIG. 7 shows the temporal profile of the renal damage inflicted by gentamicin. Significant damage only occurs after 4 days of treatment, as revealed by the evolution of serum creatinine, NAG excretion, proteinuria, and the urinary level of three sensitive markers of renal injury, such as KIM-1, NGAL and plasminogen activator inhibitor 1 (PAI-1). Congruently with the accumulated knowledge (44), serum creatinine is the least sensitive of all the markers tested. Furthermore, histological analysis of renal sections after 3 days of treatment reveals no findings of tubular damage. At this time point, cytoplasmic vacuolation of tubule epithelial cells is evident, probably resulting from the elsewhere reported accumulation of gentamicin in the endosomal compartment (45,46), and of alteration of the endocytic pathway and the endosomal trafficking (47,48). In this scenario, Western blot analysis showed that reg IIIb appears in the urine along with most other sensitive markers of renal injury, starting on day 4. Interestingly, urinary gelsolin (the ˜43 kDa fragment) appears as early as on day 1 and stays high through the treatment, long before all other sensitive markers do, including KIM-1, PAI-1, NGAL, and NAG.

### Example 8

### Origin of Urinary Reg IIIb and Gelsolin

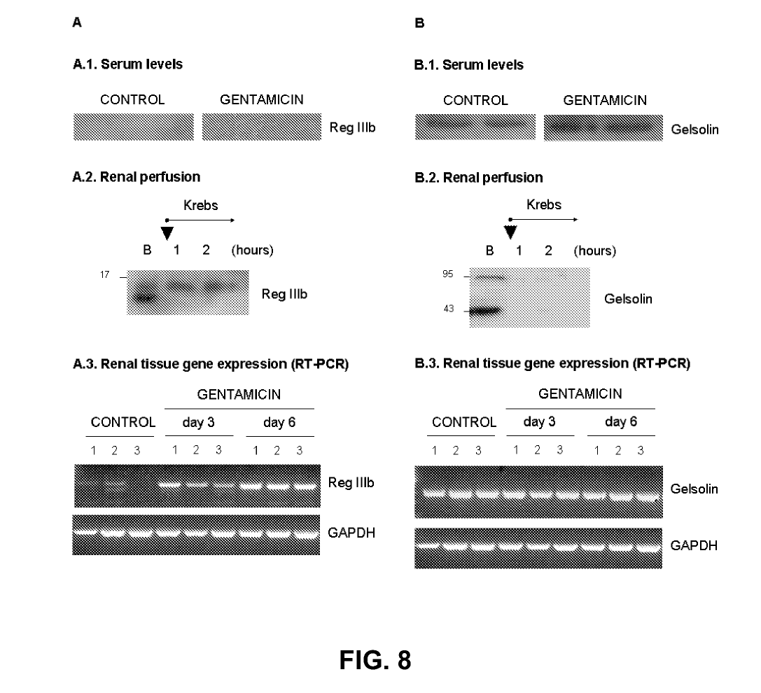

Western blot analysis of albumin-depleted serum from control and gentamicin-treated rats indicated that reg IIIb is absent (to the detection limit of this technique), whereas gelsolin is normally found in the blood compartment. Even more, gentamicin slightly increases the serum level of this latter (FIG. 8-B). Gene expression analysis carried out on renal tissue by RT-PCR showed that these 2 proteins are normally expressed in the kidneys. Treatment of rats with gentamicin does not modify the renal expression pattern of gelsolin, but induces an increase in reg IIIb gene expression as early as on day 3 (FIG. 8-A), when no detectable renal damage has occurred yet (FIG. 7). On day 6, reg IIIb expression is highest.

In order to study whether the origin of these urinary proteins was the blood, which would shed them to the urine through the glomerular filtration barrier, we perfused the kidneys of rats treated during 6 days with gentamicin with Krebs solution (containing dextran to compensate for the oncotic pressure). We found that, immediately before substituting the renal blood flow with Krebs, we could still detect reg IIIb and gelsolin in the urine (FIG. 8). However, once the renal blood flow was substituted with Krebs flow, gelsolin disappeared from the urine; yet, the upper band of reg IIIb was still detected, whereas the lower one disappeared.

### Analysis of the Results of Examples 5-8.

Nephrotoxicity poses a considerable health and economic problem worldwide. It is an important reason of failure along the drug discovery process, which leads to discarding otherwise clinically interesting molecules. Most importantly, about 25% of the 100 most used drugs in intensive care units are potentially nephrotoxic. Overall, it is estimated that nephrotoxicity is responsible for 10-20% of the acute renal failure cases. A critical aspect for the optimal clinical handling of AKI is an early diagnosis. Important progress has been made in the last decade towards an increasingly earlier detection based on novel and more sensitive urinary markers (Vaidya V S, Ferguson M A and Bonventre J V, 2008. Annu Rev Pharmacol Toxicol, 48: 463-493). However, AKI diagnosis may still be improved in an individual-drug basis, for enhanced theranostics and a more individualized medicine. In this article we provide some evidence on new urinary markers with potentially to differentiate the nephrotoxicity of gentamicin from that caused by cisplatin, and to detect the renal effects of gentamicin earlier than with state-of-the-art AKI markers. They will help to better delineate the pharmacological profile of gentamicin and, in turn, to improve its clinical utility.

Both reg IIIb and full length gelsolin have potential for a differential or aetiological diagnosis of gentamicin's nephrotoxicity. They appear in the urine of rats with overt renal failure induced by gentamicin, but are not present in the urine of rats with a similar degree of renal damage inflicted by cisplatin. Reg IIIb is a 17 kDa member of the calcium dependent lectin (C-type lectin) superfamily (Zhang Y W, Ding L S and Lai M D, 2003. World J Gastroenterol, 9: 2635-2641) comprising several secretory protein products of four genes (Reg I, II, III and IV). Reg genes have been found in different mammal species including human, rat and mouse. Rat Reg genes map to the 4q33-q34 chromosomal region. In humans, all Reg genes except Reg IV, map to the 2p12 region. In general terms, Reg family proteins are involved in tissue regeneration in a number of physiological and pathological situations, most prominently including pancreatitis, but also hepatic injury, diabetes and cancer (Zhang Y W, Ding L S and Lai M D, 2003. World J Gastroenterol, 9: 2635-2641). Our experiments suggest that reg IIIb may be implicated in renal tissue injury and repair during gentamicin treatment and, importantly, that it might be used as a differential urinary marker. Because we could not detect this protein in the serum, we thought that urinary reg IIIb may be originated in the renal tissue. Indeed, our data indicates that Reg IIIb expression is strongly induced by gentamicin in the kidneys, even preceding urine and serum markers and histological findings of nephrotoxicity (on day 3 of treatment; FIG. 8-A). The renal origin of reg IIIb upon treatment with gentamicin is further supported by our experiments on renal perfusion. When we acutely substituted the renal blood flow for perfused Krebs solution in rats previously treated with gentamicin, we still observed reg IIIb in the urine (FIG. 8-A.2). Urinary reg IIIb appears as a double band in Western blot analysis, corresponding to a double spot in 2D gels. However, when blood is substituted for Krebs in the renal circuit of gentamicin-treated rats, only the upper band is detected in the urine. We can only speculate that the lower band corresponds to a proteolytic fragment produced by serum proteases, or renal proteases activated by serum components.

Gelsolin is a highly conserved 82 kDa protein of the gelsolin superfamily. It is involved in cytoskeleton organization and rearrangement in a number of normal cellular processes including motility, signalling and apoptosis (Kwiatkowski D J, 1999. Curr Opin Cell Biol, 11: 103-108); and pathophysiological conditions, such as inflammation, cancer and amyloidosis (Spinardi L and Witke W, 2007. Subcell Biochem, 45: 55-69). Gelsolin is expressed in many cell types and is also secreted and found normally in the blood of vertebrates. Gelsolin is a known substrate for effector caspase 3, which yields a 42 kDa proteolytic fragment (t-gelsolin; Sakurai N and Utsumi T, 2006. J Biol Chem, 281: 14288-14295) involved in the execution and regulation of apoptosis. Our results indicate that urinary gelsolin may also be developed as a marker for the differential diagnosis of gentamicin's nephrotoxicity. In fact, the band corresponding to the full length protein in Western blot studies appears in the urine of gentamicin-treated rats, but it is mostly absent in cisplatin-treated rats. On the contrary, the ˜43 kDa band in our gels, likely t-gelsolin, is common to both gentamicin and cisplatin groups. The results shown in FIG. 8 indicate that gelsolin gene expression is not modified in the kidneys of rats treated with gentamicin (with respect to controls). They further show that gelsolin disappears from the urine when renal blood flow is substituted for Krebs, suggesting that urinary gelsolin is probably filtered from the blood through the glomerular filtration barrier. This may also explain why the full length gelsolin is detected in the urine of rats treated with gentamicin and not in those treated with cisplatin, whereas t-gelsolin appears after both treatments. Gentamicin alters the GFB properties leading to increase filtration of specific proteins. The polycationic charge of gentamicin alters the electrostatic properties of the GFB, increasing the permeability of negatively charged proteins (such as gelsolin, pl=5.75) and lowering the sieving coefficient of positively charged ones (Cojocel et al., 1983. Toxicol Appl Pharmacol, 68: 96-109). No alterations of the GFB sieving properties have been reported for cisplatin. In this case, full length gelsolin would be excluded from passing through the GFB for size restriction. However, t-gelsolin would not be trapped in the blood in any case because its lower size allows it to filter more easily through the GFB. In a scenario of tubular necrosis, a larger amount of proteins scapes the handicapped tubular reabsorption capacity, which can be detected in the urine. As shown in FIG. 7, t-gelsolin appears in the urine of gentamicin-treated rats significantly earlier than traditional and new AKI markers, the latter including KIM-1, NGAL, NAG and PAI-1. This might also be exploited for an early monitoring of gentamicin's nephrotoxicity.

The present study provides two novel urinary biomarker candidates for the differential diagnosis of gentamicin's nephrotoxicity, which need to be further developed in the preclinical and clinical settings for a better theranostic usage and efficacy of this drug. Moreover, it poses a proof-of-principle for the potential application of the aetiological diagnosis of AKI to critical patients coursing with multiple conditions potentially affecting renal integrity, including polymedication. Aetiological diagnosis should be extended on many other potentially nephrotoxic drugs widely used in the clinical practice, and on pre-renal and post-renal causes of AKI. This will enable us to delineate patterns of markers that specifically discriminate the origin of undesirable renal effects in order to appropriately and selectively reshape the clinical handling and therapeutic regimes of patients at risk.

1-56. (canceled)

57. A method to provide useful data for determining renal damage or the risk to suffer it, comprising:
a. obtaining an isolated biological sample from an individual, and
b. detecting and/or quantifying, in the sample obtained in (a), the fragment t-gelsolin of the protein gelsolin having about 43 KDa; a gelsolin at least 80% identical to SEQ ID NO: 5; a Reg3B protein at least 60% identical to SEQ ID NO: 1; or any combination thereof,
wherein the renal damage is due to the administration of or exposure to at least one nephrotoxic agent.

58. The method according to claim 57, which additionally comprises comparing the data obtained in step (b) with data obtained from at least one control sample in order to find any significant deviation.

59. The method according to claim 58, wherein in addition it comprises the attribution of the significant deviation to the development of said renal damage in the individual.

60. The method according to claim 57, wherein the nephrotoxic agent is an aminoglycoside antibiotic.

61. The method according to claim 57, wherein the nephrotoxic agent is gentamicin.

62. The method according to claim 57, wherein the fragment t-gelsolin; the gelsolin at least 80% identical to SEQ ID NO: 5; the Reg3B protein at least 60% identical to SEQ ID NO: 1; or any combination thereof, is detected and/or quantified from 12 hours or from 24 hours after the beginning of the administration of or exposure to an aminoglycoside antibiotic as the nephrotoxic agent.

63. The method according to claim 57, wherein the t-gelsolin is detected and/or quantified from 12 hours or from 24 hours after the beginning of the administration of or exposure to the nephrotoxic agent cisplatin.

64. The method according to claim 57, wherein according with the step (b) it is further detected and/or quantified at least one protein, or any combination thereof, selected from the list comprising:
a protein having at least 60% identity in respect of amino acid sequence SEQ ID NO: 2,
a protein having at least 80% identity in respect of amino acid sequence SEQ ID NO: 3-4, 6-11,
a protein having at least 90% identity in respect of amino acid sequence SEQ ID NO: 12 to 14.

65. The method according to claim 57, for additionally determining the cause of the renal damage, differentiating said renal damage caused by gentamicin from that caused by cisplatin, wherein it is detected and/or quantified t-gelsolin and:
i. if it is detected and/or quantified the gelsolin at least 80% identical to SEQ ID NO: 5 and/or the Reg3B protein at least 60% identical to SEQ ID NO: 1, the cause of the renal damage is the administration or exposure to gentamicin,
ii. if it is not detected the gelsolin at least 80% identical to SEQ ID NO: 5 and/or the Reg3B protein at least 60% identical to SEQ ID NO: 1, the cause of the renal damage is the administration or exposure to cisplatin.

66. The method according to claim 57, for predicting the progression of renal damage due to the administration of or exposure to at least one nephrotoxic agent, comprising:
i. determining a first concentration; of the fragment t-gelsolin; of the gelsolin at least 80% identical to SEQ ID NO: 5; of the Reg3B protein at least 60% identical to SEQ ID NO: 1; or any combination thereof; or further determining the concentration of any of the proteins selected from the list comprising
a protein having at least 60% identity in respect of amino acid sequence SEQ ID NO: 2,
a protein having at least 80% identity in respect of amino acid sequence SEQ ID NO: 3-4, 6-11,
a protein having at least 90% identity in respect of amino acid sequence SEQ ID NO: 12 to 14,
in combination with t-gelsolin, said gelsolin or said Reg3B, or any combinations thereof; in an isolated biological sample from an individual exposed or not exposed to a nephrotoxic agent,
ii. determining a second concentration of any t-gelsolin or the protein/s from step (i) in the isolated sample from the individual, after determining the first concentration of the fragment and/or protein/s in the exposed individual, or after initiation of administration or exposure to the nephrotoxic agent in the not exposed individual, and
iii. comparing the second concentration obtained in step (ii) with the first concentration obtained in step (i) to find any significant deviation.

67. The method according to claim 66, wherein the nephrotoxic agent is an aminoglycoside antibiotic.

68. The method according to claim 67, wherein the nephrotoxic agent is gentamicin.

69. The method according to claim 57, wherein the renal damage is acute renal failure.

70. The method according to claim 57, wherein the biological sample from step (a) is a bodily fluid.

71. The method according to claim 70, wherein the bodily fluid is urine or serum.

72. The method according to claim 57, wherein the individual is a human.

73. The method according to claim 57, wherein the fragment t-gelsolin; the gelsolin at least 80% identical to SEQ ID NO: 5; the Reg3B protein at least 60% identical to SEQ ID NO: 1; or any combination thereof, is detected and/or quantified by means of a kit comprising reagents for detecting any of said fragment or protein's.

74. The method according to claim 57, wherein the fragment t-gelsolin; the gelsolin at least 80% identical to SEQ ID NO: 5; the Reg3B protein at least 60% identical to SEQ ID NO: 1; or any combination thereof, is detected and/or quantified by means of a kit comprising one or more probes that recognise any of said fragment or protein's.

75. The method according to claim 74, wherein said probe/s are attached to a solid support.

76. The method according to claim 57, wherein the fragment t-gelsolin; the gelsolin at least 80% identical to SEQ ID NO: 5; the Reg3B protein at least 60% identical to SEQ ID NO: 1; or any combination thereof, is detected and/or quantified by means of a kit comprising antibodies that recognise any of said fragment or protein's.


SEQUENCE LISTING (20 sequences)

SEQ ID NO: 1 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 2 - protein, 382 aa, Homo sapiens
MGLLLPLALCILVLCCGAMSPPQLALNPSALLSRGCNDSDVLAVAGFALRDINKDRKDGYVLRLNRVNDAQEYRRGGLGSLFYLTLDVLETDCHVLRKKAWQDCGMRIFFESVYGQCKAIFYMNNPSRVLYLAAYNCTLRPVSKKKIYMTCPDCPSSIPTDSSNHQVLEAATESLAKYNNENTSKQYSLFKVTRASSQWVVGPSYFVEYLIKESPCTKSQASSCSLQSSDSVPVGLCKGSLTRTHWEKFVSVTCDFFESQAPATGSENSAVNQKPTNLPKVEESQQKNTPPTDSPSKAGPRGSVQYLPDLDDKNSQEKGPQEAFPVHLDLTTNPQGETLDISFLFLEPMEEKLVVLPFPKEKARTAECPGPAQNASPLVLPP

SEQ ID NO: 3 - protein, 413 aa, Homo sapiens
MAFIAALGLLMAGICPAVLCDGTLGRDTLSHEDHGKGRQLHSLTLASSNTDFALSLYKKLALRNPDKNVVFSPLSISAALTILSLGAKDSTMEEILEGLKFNLTEITEEEIHQGFGHLLQRLSQPEDQVEINTGSALFIDKEQPILSEFQEKTRALYQAEAFVADFKQPNEAKKLINDYVSNQTQGKIAELFSDLEERTSMVLVNYLLFKGKWKAPFNPNDTIESEFYLDEKRSVKVPMMKIKEVTTPYVRDEELSCSVLELKYTGNASALFILPDQGKMQQVESSLQPETLKKWKDSLIPRIINDLRMPKFSISTDYSLKEVLPELGIKKVFSQQADLSRITGTKDLYVSQVVHKAVLDVDETGTEATAATGVATVIRRQPRTLNFNRPFMVVITDMDSQSILFVAKITNPK

SEQ ID NO: 4 - protein, 298 aa, Homo sapiens
MFSRAGVAGLSAWTLQPQWIQVRNMATLKDITRRLKSIKNIQKITKSMKMVAAAKYARAERELKPARIYGLGSLALYEKADIKGPEDKKKHLLIGVSSDRGLCGAIHSSIAKQMKSEVATLTAAGKEVMLVGIGDKIRGILYRTHSDQFLVAFKEVGRKPPTFGDASVIALELLNSGYEFDEGSIIFNKFRSVISYKTEEKPIFSLNTVASADSMSIYDDIDADVLQNYQEYNLANIIYYSLKESTTSEQSARMTAMDNASKNASEMIDKLTLTFNRTRQAVITKELIEIISGAAALD

SEQ ID NO: 5 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 6 - protein, 137 aa, Homo sapiens
MSSLIRRVISTAKAPGAIGPYSQAVLVDRTIYISGQIGMDPSSGQLVSGGVAEEAKQALKNMGEILKAAGCDFTNVVKTTVLLADINDFNTVNEIYKQYFKSNFPARAAYQVAALPKGSRIEIEAVAIQGPLTTASL

SEQ ID NO: 7 - protein, 408 aa, Homo sapiens
MTSKGPEEEHPSVTLFRQYLRIRTVQPKPDYGAAVAFFEETARQLGLGCQKVEVAPGYVVTVLTWPGTNPTLSSILLNSHTDVVPVFKEHWSHDPFEAFKDSEGYIYARGAQDMKCVSIQYLEAVRRLKVEGHRFPRTIHMTFVPDEEVGGHQGMELFVQRPEFHALRAGFALDEGIANPTDAFTVFYSERSPWWVRVTSTGRPGHASRFMEDTAAEKLHKVVNSILAFREKEWQRLQSNPHLKEGSVTSVNLTKLEGGVAYNVIPATMSASFDFRVAPDVDFKAFEEQLQSWCQAAGEGVTLEFAQKWMHPQVTPTDDSNPWWAAFSRVCKDMNLTLEPEIMPAATDNRYIRAVGVPALGFSPMNRTPVLLHDHDERLHEAVFLRGVDIYTRLLPALASVPALPSDS

SEQ ID NO: 8 - protein, 336 aa, Homo sapiens
GTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 9 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 10 - protein, 110 aa, Homo sapiens
MSMTDLLNAEDIKKAVGAFSATDSFDHKKFFQMVGLKKKSADDVKKVFHMLDKDKSGFIEEDELGFILKGFSPDARDLSAKETKMLMAAGDKDGDGKIGVDEFSTLVAES

SEQ ID NO: 11 - protein, 119 aa, Homo sapiens
QDGMYQRFLRQHVHPEETGGSDRYCDLMMQRRKMTLYHCKRFNTFIHEDIWNIRSICSTTNIQCKNGKMNCHEGVVKVTDCRDTGSSRAPNCRYRAIASTRRVVIACEGNPQVPVHFDG

SEQ ID NO: 12 - protein, 413 aa, Homo sapiens; identical to SEQ ID NO: 3

SEQ ID NO: 13 - protein, 491 aa, Homo sapiens
MPLPVQVFNLQGAVEPMQIDVDPQEDPQNAPDVNYVVENPSLDLEQYAASYSGLMRIERLQFIADHCPTLRVEALKMALSFVQRTFNVDMYEEIHRKLSEATRSSLRELQNAPDAIPESGVEPPALDTAWVEATRKKALLKLEKLDTDLKNYKGNSIKESIRRGHDDLGDHYLDCGDLSNALKCYSRARDYCTSAKHVINMCLNVIKVSVYLQNWSHVLSYVSKAESTPEIAEQRGERDSQTQAILTKLKCAAGLAELAARKYKQAAKCLLLASFDHCDFPELLSPSNVAIYGGLCALATFDRQELQRNVISSSSFKLFLELEPQVRDIIFKFYESKYASCLKMLDEMKDNLLLDMYLAPHVRTLYTQIRNRALIQYFSPYVSADMHRMAAAFNTTVAALEDELTQLILEGLISARVDSHSKILYARDVDQRSTTFEKSLLMGKEFQRRAKAMMLRAAVLRNQIHVKSPPREGSQGELTPANSQSRMSTNM

SEQ ID NO: 14 - protein, 313 aa, Homo sapiens
MPNTAMKKKVLLMGKSGSGKTSMRSIIFANYIARDTRRLGATIDVEHSHVRFLGNLVLNLWDCGGQDTFMENYFTSQRDNIFRNVEVLIYVFDVESRELEKDMHYYQSCLEAILQNSPDAKIFCLVHKMDLVQEDQRDLIFKEREEDLRRLSRPLECACFRTSIWDETLYKAWSSIVYQLIPNVQQLEMNLRNFAQIIEADEVLLFERATFLVISHYQCKEQRDVHRFEKISNIIKQFKLSCSKLAASFQSMEVRNSNFAAFIDIFTSNTYVMVVMSDPSIPSAATLINIRNARKHFEKLERVDGPKHSLLMR

SEQ ID NO: 15 - DNA, 20 bp, Rattus norvegicus
ttgtttgatgcagaactggc

SEQ ID NO: 16 - DNA, 20 bp, Rattus norvegicus
agacgtagggcaacttcact

SEQ ID NO: 17 - DNA, 20 bp, Rattus norvegicus
caaggctacttcaagtctgg

SEQ ID NO: 18 - DNA, 20 bp, Rattus norvegicus
gctaccctcttcagacacat

SEQ ID NO: 19 - DNA, 19 bp, Rattus norvegicus
gtggtcatgagcccttcca

SEQ ID NO: 20 - DNA, 23 bp, Rattus norvegicus
aactccctcaagattgtcagcaa